(19) United States
(12) Patent Application Publication
(10) Pub. No.: US 2024/0120113 A1
(43) Pub. Date: Apr. 11, 2024

(54) PROSPECTING BIOMEDICAL INFORMATION

(71) Applicant: Scinapsis Analytics Inc., dba BenchSci, Toronto (CA)

(72) Inventors: Casandra Savitri MANGROO, Pickering (CA); Luigi GENTILE, Mississauga (CA)

(21) Appl. No.: 18/379,077

(22) Filed: Oct. 11, 2023

Related U.S. Application Data
(63) Continuation-in-part of application No. 17/958,142, filed on Sep. 30, 2022.
Application No. 17/958,142 is a continuation-in-part of application No. 17/958,217, filed on Sep. 30, 2022.
Application No. 17/958,217 is a continuation-in-part of application No. 17/958,196, filed on Sep. 30, 2022.

(51) Int. Cl. G16H 50/70; G16H 50/30

(52) U.S. Cl. CPC G16H 50/70; G16H 50/30

(57) ABSTRACT

A method implements the prospecting of biomedical information. The method includes processing multiple result graphs using a target identifier and a disease identifier to generate a biology score value representing a link between the target identifier and the disease identifier. The method further includes processing the result graphs using a therapeutic identifier and the target identifier to generate a therapeutic score value representing a link between the therapeutic identifier and the target identifier. The method further includes processing the result graphs using the therapeutic identifier, the target identifier, and the disease identifier to generate a liability score value representing a link between one or more risk tags and the therapeutic identifier. The method further includes processing the biology, therapeutic, and liability score values to generate combined score value representing a likelihood that a disease identified by the disease identifier is treatable with a therapeutic identified with the therapeutic identifier.

### CROSS REFERENCE TO RELATED APPLICATIONS

This application is a continuation in part of U.S. patent application Ser. No. 17/958,142, filed Sep. 30, 2022. This patent application is a continuation in part of U.S. patent application Ser. No. 17/958,217, filed Sep. 30, 2022. This patent application is a continuation in part of U.S. patent application Ser. No. 17/958,196, filed Sep. 30, 2022. Each of the patent applications listed above are hereby incorporated by reference herein.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of biomedical information provide access to the electronic forms of the publications. A challenge is for computing systems to automatically determine and display values pertaining to experiments and projects relating to biomedical information.

### SUMMARY

In general, in one or more aspects, the disclosure relates to a method implementing the prospecting of biomedical information. The method includes processing multiple result graphs using a target identifier and a disease identifier to generate a biology score value representing a link between the target identifier and the disease identifier. The method further includes processing the result graphs using a therapeutic identifier and the target identifier to generate a therapeutic score value representing a link between the therapeutic identifier and the target identifier. The method further includes processing the result graphs using the therapeutic identifier, the target identifier, and the disease identifier to generate a liability score value representing a link between one or more risk tags and the therapeutic identifier. The method further includes processing the biology score value, the therapeutic score value, and the liability score value to generate combined score value representing a likelihood that a disease identified by the disease identifier is treatable with a therapeutic identified with the therapeutic identifier. The method further includes presenting the combined score value. The combined score value may be displayed on a user device.

In general, in one or more aspects, the disclosure relates to a system implementing the prospecting of biomedical information. The system includes at least one processor and an application executing on the at least one processor. The application performs processing multiple result graphs using a target identifier and a disease identifier to generate a biology score value representing a link between the target identifier and the disease identifier. The application further performs processing the result graphs using a therapeutic identifier and the target identifier to generate a therapeutic score value representing a link between the therapeutic identifier and the target identifier. The application further performs processing the result graphs using the therapeutic identifier, the target identifier, and the disease identifier to generate a liability score value representing a link between one or more risk tags and the therapeutic identifier. The application further performs processing the biology score value, the therapeutic score value, and the liability score value to generate combined score value representing a likelihood that a disease identified by the disease identifier is treatable with a therapeutic identified with the therapeutic identifier. The application further performs presenting the combined score value. The combined score value may be displayed on a user device.

In general, in one or more aspects, the disclosure relates to a non-transitory computer readable storage medium storing computer readable program code which, when executed by a processor implements the prospecting of biomedical information. The code performs processing multiple result graphs using a target identifier and a disease identifier to generate a biology score value representing a link between the target identifier and the disease identifier. The code further performs processing the result graphs using a therapeutic identifier and the target identifier to generate a therapeutic score value representing a link between the therapeutic identifier and the target identifier. The code further performs processing the result graphs using the therapeutic identifier, the target identifier, and the disease identifier to generate a liability score value representing a link between one or more risk tags and the therapeutic identifier. The code further performs processing the biology score value, the therapeutic score value, and the liability score value to generate combined score value representing a likelihood that a disease identified by the disease identifier is treatable with a therapeutic identified with the therapeutic identifier. The code further performs presenting the combined score value. The combined score value may be displayed on a user device.

Other aspects of the one or more embodiments will be apparent from the following description and the appended claims.

### BRIEF DESCRIPTION OF DRAWINGS

FIG. 1, FIG. 2A, FIG. 2B, and FIG. 3 show diagrams of systems in accordance with disclosed embodiments.

FIG. 4 shows a flowchart in accordance with disclosed embodiments.

FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21, and FIG. 22 show examples in accordance with disclosed embodiments.

FIG. 23A and FIG. 23B show computing systems in accordance with disclosed embodiments.

Similar elements in the various figures are denoted by similar names and reference numerals. The features and elements described in one figure may extend similarly named features and elements in different figures.

### DETAILED DESCRIPTION

Embodiments of the disclosure prospect biomedical information to automatically determine and display values pertaining to experiments and projects relating to biomedical information. For example, Wendy, a user of the system, decides to analyze a biomedical project (referred to as a project). Instead of performing the analysis herself, Wendy opens a website to view information about the project. In response to receiving the request from Wendy, the system may automatically generate scores and values that are displayed to the device Wendy is using. The scores and values may identify the likelihood that a target (such as a gene, a protein, a pathway, etc.) affects a disease, the likelihood that a therapeutic (such as a drug treatment regimen, a medication, etc.) affects the target, and a likelihood that a risk may be associated with the target and the therapeutic. These values may be combined into a combined value that provides an analysis for a project.

Additionally, project data and accounting data may be analyzed to identify costs and revenue projections for the project that relate to the therapeutic (as well as the target and the disease) to generate a pharmaceutical net present value in which the interest rate used to discount the projections is related to the likelihood of success of the project.

Additionally, the system may provide summaries generated from machine learning models. System generates result graphs from files (e.g., publications) of biomedical information that describe experiments related to various targets, diseases, therapeutics, etc. The result graphs may be converted to input for language models that output summaries describing the relevant information from the file that relates to a project.

Turning to FIG. 1, the system (100) performs prospecting of biomedical information. The system (100) converts biomedical information from files to result graphs and generates risk tags. The result graphs and risk tags are used to generate values to analyze biomedical projects. The system (100) receives requests (e.g., the request (118)) and generates responses (e.g., the response (125)) using the result graphs A (120) and the risk tags A (121). The system (100) generates the result graphs A (120) from biomedical information (e.g., the files (130)) stored in the file data (155) using multiple machine learning and natural language processing models. The system (100) generates the risk tags A (121) from the result graphs A (120). The system (100) uses the result graphs A (120) and the risk tags A (121) to generate the response (125). The system (100) may display the result graphs A (120), the images from the files of the file data (155), and values generated from the result graphs A (120) and the risk tags A (121) to users operating the user devices A (102) and B (107) through N (109). The system (100) includes the user devices A (102) and B (107) through N (109), the server (112), and the repository (150).

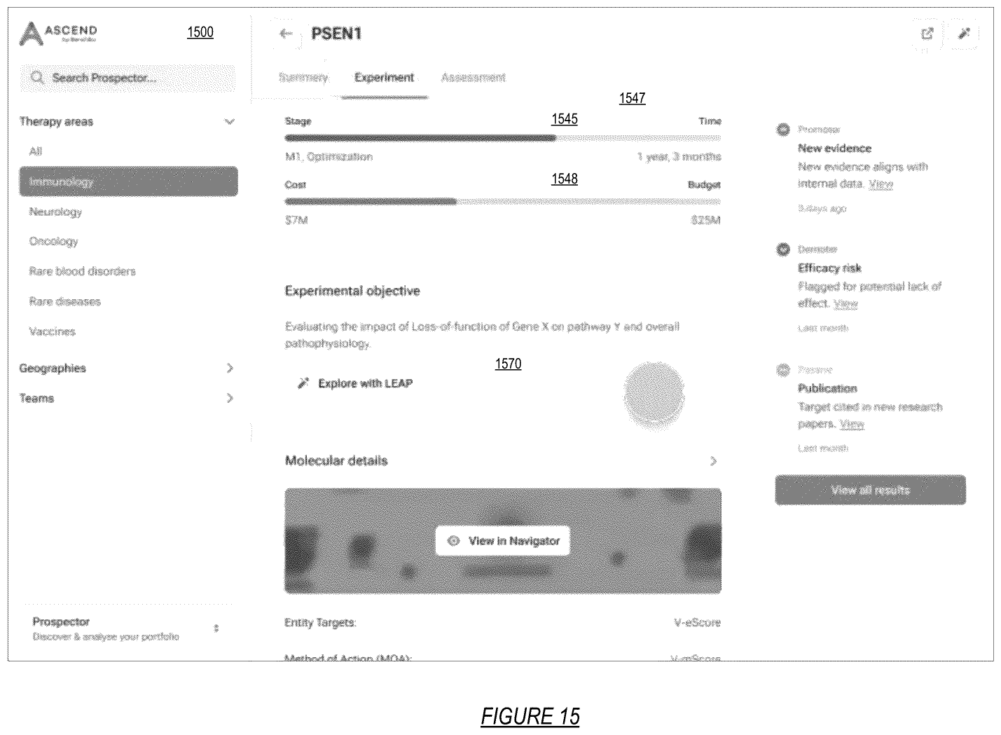

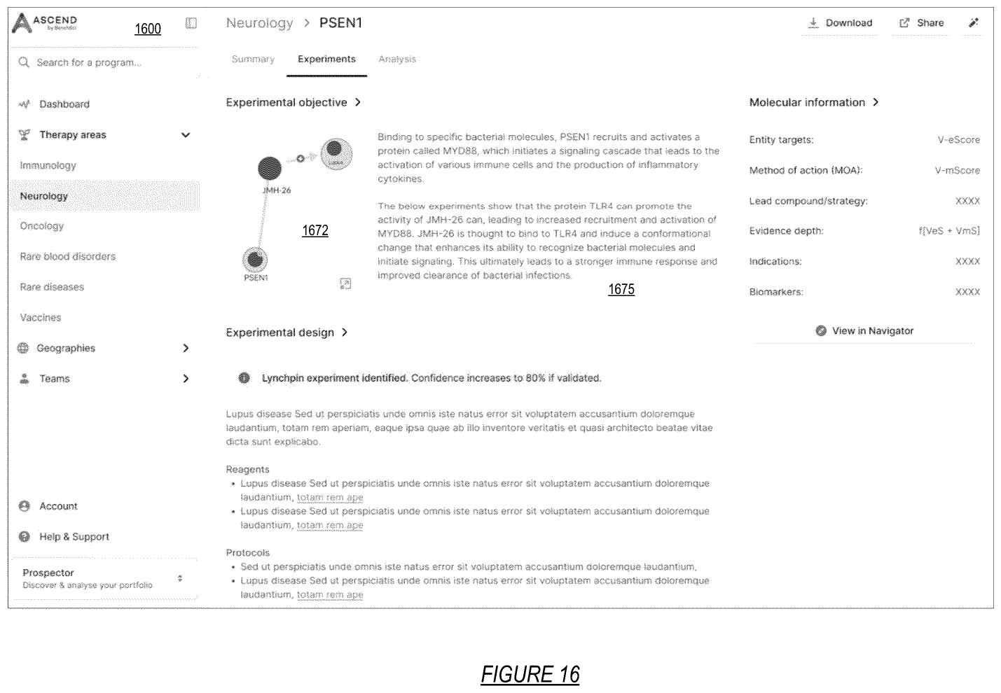

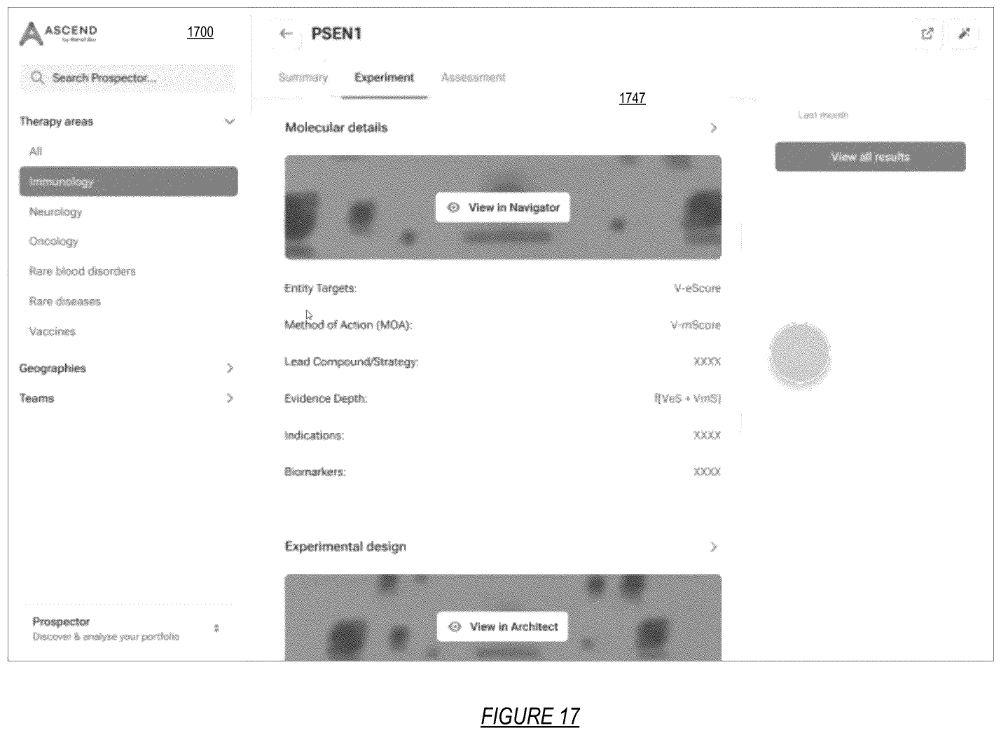

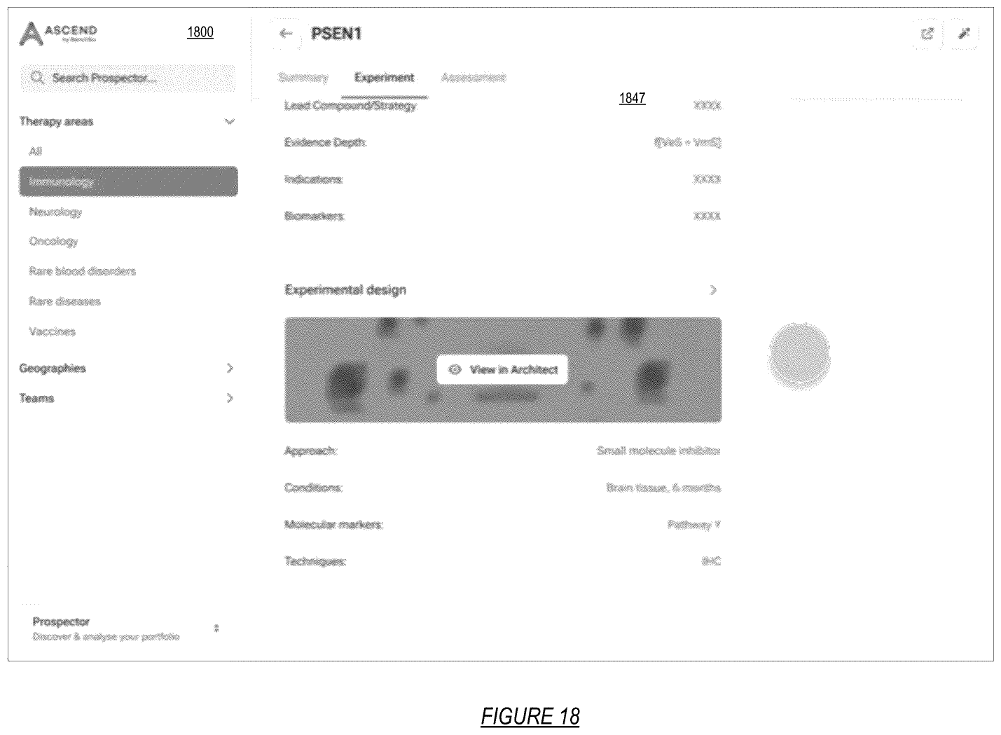

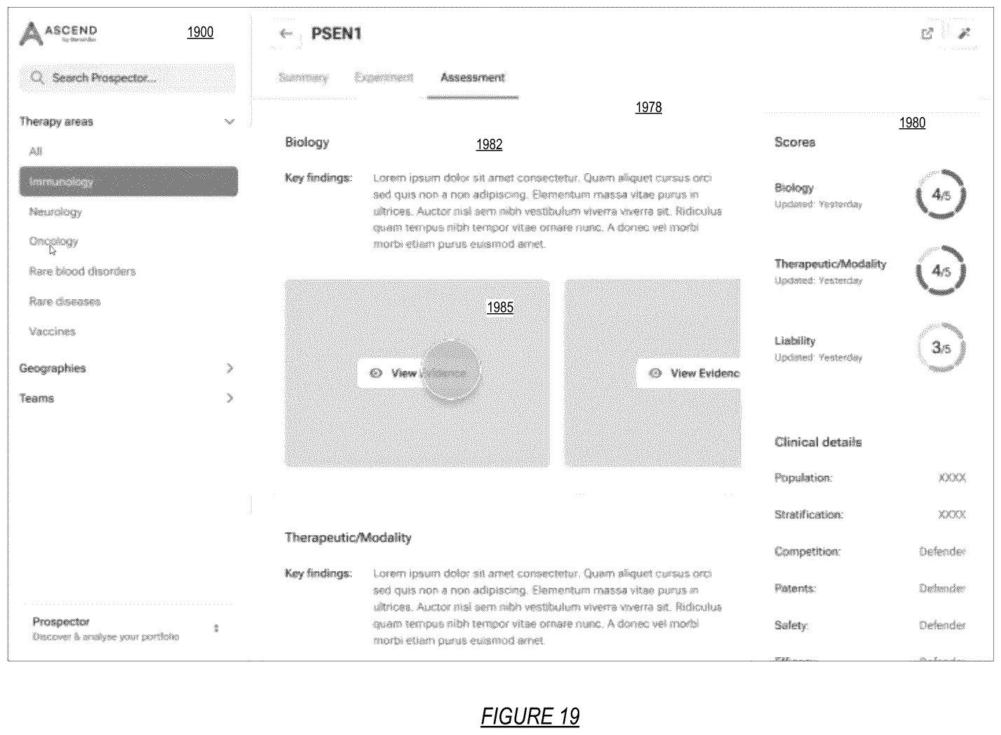

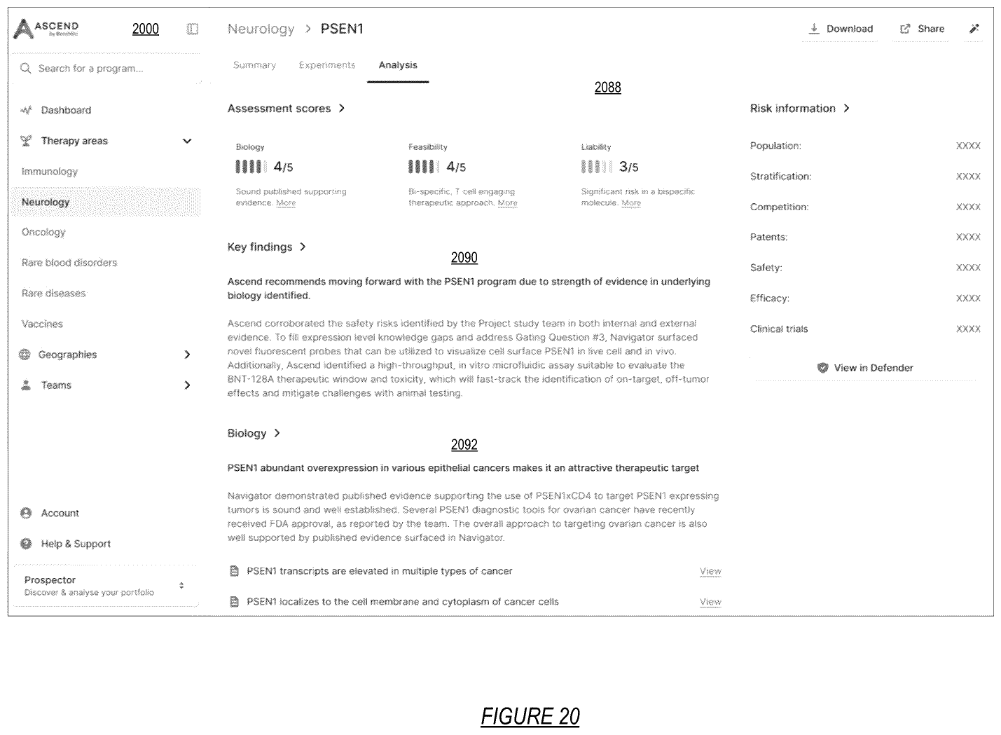

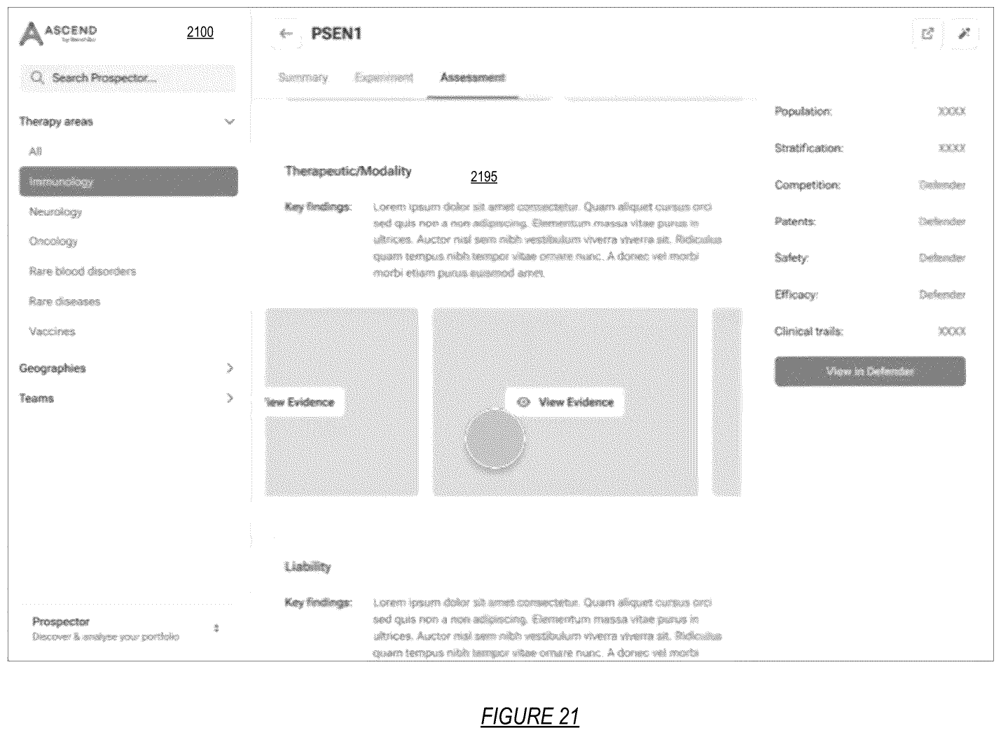

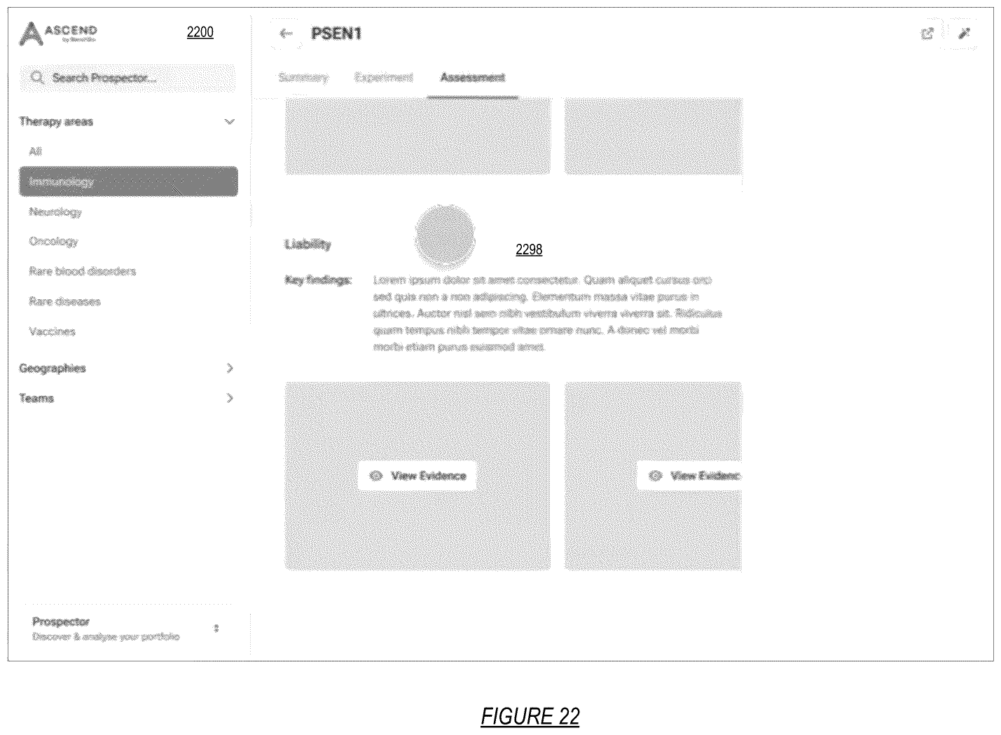

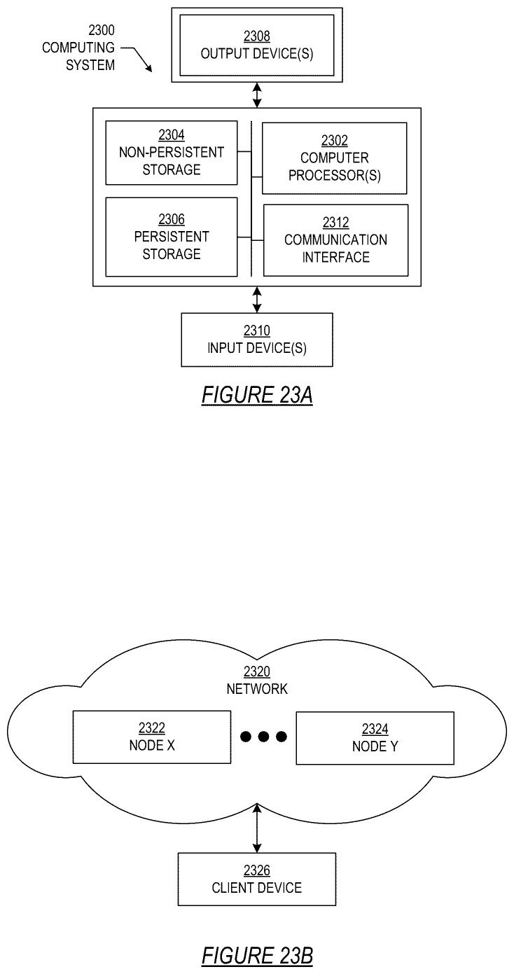

The server (112) is a computing system (further described in FIG. 23A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the result graphs A (120) using the interface controller (122). The server application (115) may host websites accessed by users of the user devices A (102) and B (107) through N (109) to view information from the result graphs A (120) and the file data (155). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the result graphs A (120).

The request (118) is a request from one of the user devices A (102) and B (107) through N (109). In one embodiment, the request (118) is a request for information about a project that uses one or more entities defined in the ontology library (152), described in the file data (155), and graphed in the graph data (158). In one embodiment, the request (118) may include target identifiers, disease identifiers, and therapeutic identifiers related to one or more projects. The structured text below (formatted in accordance with JSON) provides an example of entities with identifiers (universally unique identifiers (UUIDs) that may be specified in the request (118) using key value pairs.

```
{
  “project”: “Project A”,
  ″target″:
  {
    “UUID”: “4ea4221f-9b02-4d3c-b7a6-01c904a6e4a7”,
    “entity type”: “protein”,
    “entity”: “BRD9”
  },
  ″disease″:
  {
    “UUID”: “8b8b6b9e-7a52-4f56-ae75-9c2d8f0d1e9d”,
    “entity type”: “disease”,
    “entity”: “breast cancer”
  },
  ″therapeutic″:
  {
    “UUID”: “c10aee4f-4b13-471a-93d3-29c7d4e4c259”,
    “entity type”: “medication”,
    “entity”: “Pertuzumoxifen”
  }
}
```

In one embodiment, the UUIDs for the target identifier, the disease identifier, and the therapeutic identifier are in the same identifier space. In one embodiment, the UUIDs correspond to 128 bits written as 32-character hexadecimal string separated by hyphens. Different types of identifiers with different textual representations may be used.

The result graphs A (120) and the risk tags A (121) are generated with the modeling application (128), described further below. The result graphs A (120) and the risk tags A (121) may be subsets of the result graphs B (135) and the risk tags B (133), respectively. The result graphs A (120) includes nodes and edges in which the nodes correspond to text from the file data (155) and the edges correspond to semantic relationships between the nodes. The result graphs A (120) are directed graphs in which the edges identify a direction from one node to a subsequent node in the result graphs A (120). In one embodiment, the result graphs A (120) are acyclic graphs. The result graphs A (120) may be stored in the graph data (158) of the repository (150). The risk tags A (121) may be stored in the risk data (156) of the repository (150).

The interface controller (122) is a collection of programs that may operate on the server (112). The interface controller (122) processes the request (118) using the result graphs A (120) and the risk tags (121) to generate the response (125). In one embodiment, the interface controller (122) searches the graph data (158) to identify the result graphs A (120) (which may include some of the result graphs from the result graphs B (135)) that include information about the entities identified in the request (118).

The project value controller (142) is a collection of programs that may operate on the server (112). Responsive to the request (118), the project value controller (142) generates a net present value for the project identified in the request (118). Output from the project value controller (142) may be stored in the analysis data (153) of the repository (150).

The scoring controller (143) is a collection of programs that may operate on the server (112). Responsive to the request (118), the scoring controller (143) generates values that form an analysis of the likelihood of success of the project identified in the request (118). Output from the scoring controller (143) may be stored in the analysis data (153) of the repository (150).

The summary controller (144) is a collection of programs that may operate on the server (112). Responsive to the request (118), the summary controller (144) generates text that may summarize information from files stored in the file data (155) that is related to the targets, diseases, and therapeutics identified in the request (118). Output from the summary controller (144) may be stored in the analysis data (153) of the repository (150).

The response (125) is generated by the interface controller (122) in response to the request (118) using the result graphs A (120). In one embodiment, the response (125) includes the values and text generated by the project value controller (142), the scoring controller (143), and the summary controller (144). The response (125) may further include one or more of the result graphs A (120) and information from the file data (155). Portions of the response (125) may be displayed by the user devices A (102) and B (107) through N (109) that receive the response (125).

The modeling application (128) is a collection of programs that may operate on the server (112). The modeling application (128) generates the result graphs B (135) from the files (130) using a result graph controller (132).

The files (130) include biomedical information and form the basis for the result graphs B (135). The files (130) include the file (131), which is the basis for the result graph (137). Each file includes multiple sentences and may include multiple images of evidence. The evidence may identify how different entities, defined in the ontology library (152), affect each other. For example, entities that are proteins may suppress or enhance the expression of other entities and affect the prevalence of certain diseases. Types of entities include proteins, genes, diseases, experiment techniques, chemicals, cell lines, pathways, tissues, cell types, organisms, etc. In one embodiment, nouns and verbs from the sentences of the file (131) are mapped to the result nodes (138) of the result graph (137). In one embodiment, the semantic relationships between the words in the sentences corresponding to the result nodes (138) are mapped to the result edges (140). In one embodiment, one file serves as the basis for multiple result graphs. In one embodiment, one sentence from a file may serve as the basis for one result graph.

The result graph controller (132) generates the result graphs B (135) from the files (130). The result graph controller (132) is a collection of programs that may operate on the server (112). For a sentence of the file (131), the result graph controller (132) identifies the result nodes (138) and the result edges (140) for the result graph (137).

The result graphs B (135) are generated from the files (130) and includes the result graph (137), which corresponds to the file (131). The result nodes (138) represent nouns and verbs from a sentence of the file (131). The result edges (140) identify semantic relationships between the words represented by the result nodes (138).

The risk controller (136) is a collection of programs that may operate on the server (112). The risk controller (136) generates the risk tags B (133) from one or more of the file (131) and the result graph (137).

In one embodiment, the risk controller (136) uses risk signatures to identify risk events from one or more of the file (131) and the result graph (137). For example, a risk signature may identify a compound (a chemical, a protein, etc.) and a usage of the compound in a target (cell, tissue, organism, etc.). The compound and its usage may correspond to nodes of the result nodes (138). In one embodiment, the nodes for the compound and its usage are adjacent nodes.

In one embodiment, the risk controller (136) uses a machine learning model to identify risk events from the file (131). The machine learning models may take text from the file (131) (e.g., a sentence of a publication) as input and output a classification of a risk type. The modeling application (128) may process the output to generate the risk tags B (133).

The risk tags B (133) are tags that identify one or more of the files (130) and the result graphs B (135) as including a risk event. The risk tag may identify a term, phrase, or sentence from, e.g., the file (131) that correspond to and describe the risk event of the result graph (137). The risk tag may identify one or more of the result nodes (138) and result edges (140) that correspond to the risk event. The risk tags B (133) includes the risk types (134).

The risk types (134) identify the types of the risk of the risk tags B (133). Risk types include safety risks and efficacy risks. A safety risk is a risk in which an adverse event was observed that affected the safety of the cell, tissue, organ, organism, etc. For example, a safety risk may be identified in biomedical information describing an experiment in which a chemical introduced into a cell killed the cell. An efficacy risk is a risk in which an adverse event was observed that reduced the efficacy of a biomedical agent. For example, a chemical may be introduced that reduces the expression of a protein and reduces the efficacy of treatments with the protein.

The user devices A (102) and B (107) through N (109) are computing systems (further described in FIG. 23A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information including information from the graph data (158), the file data (155), and the analysis data (153). The user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to view information related to a project. Corresponding information from the graph data (158), the analysis data (153), etc., may be generated and included in the response (125) and displayed in a user interface of the user application A (105).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system described below in FIGS. 23A and 23B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the ontology library (152), the file data (155), the model data (157), the graph data (158), the risk data (156), and the analysis data (153).

The ontology library (152) includes information on the types of entities and biomedical terms and phrases used by the system (100). Multiple terms and phrases may be used for the same entity. The ontology library (152) defines types of entities. In one embodiment, the types include the types of protein/gene, chemical, cell line, pathway, tissue, cell type, disease, organism, etc. The ontology library (152) may store the information about the entities in a database, structured text files, combinations thereof, etc.

The file data (155) is biomedical information stored in electronic records. The biomedical information describes the entities and corresponding relationships that are defined and stored in the ontology library (152). The file data (155) includes the files (130). Each file in the file data (155) may include image data and text data. The image data includes images that represent the graphical figures from the files. The text data represents the writings in the file data (155). The text data for a file includes multiple sentences that may each include multiple words that may each include multiple characters. The sentences may be stored as strings in the repository (150). In one embodiment, the file data (155) includes biomedical information stored as extensible markup language (XML) files, portable document files (PDFs). The file formats define containers for the text and images of the biomedical information describing evidence of biomedical experiments.

The model data (157) includes the data for the models used by the system (100). The models may include rules-based models and machine learning models. The machine learning models may be updated by training, which may be supervised training. The modeling application (128) may load the models from the model data (157) to generate the result graphs B (135) from the files (130).

The model data (157) may also include intermediate data. The intermediate data is data generated by the models during the process of generating the result graphs B (135) from the files (130).

The model data (157) may include the signatures, models, etc., used to identify the risk data (156). The signatures may define paths in result graphs of the graph data (158) that correspond to a risk event. The models may be machine learning models that identify risk events from biomedical information in the file data (155).

The graph data (158) is the data of the graphs (including the result graphs A (120) and B (135)) generated by the system. The graph data (158) includes the nodes and edges for the graphs. The graph data (158) may be stored in a database, structured text files, combinations thereof, etc.

The risk data (156) is the data that identifies risks of adverse events of the entities identified by the system. The risk data (156) includes risk tags (including the risk tags B (133)).

The analysis data (153) includes data generated by the project value controller (142), the scoring controller (143), and the summary controller (144). The analysis data (153) provides analysis of projects that utilize the entities from the ontology library (152).

Although shown using distributed computing architectures and systems, other architectures and systems may be used. In one embodiment, the server application (115) may be part of a monolithic application that implements evidence networks. In one embodiment, the applications and programs described above may be part of monolithic applications the functions performed by the system (100) without the server application (115).

Turning to FIG. 2A, the result graph controller (232) further describes the result graph controller (132) of FIG. 1. The result graph controller (232) processes the file (231) to generate the result graphs B (235). In one embodiment, the result graph controller (232) includes the sentence controller (260), the token controller (262), the tree controller (264), and the text graph controller (267) to process the text from the file (231) describing biomedical experiments. In one embodiment, the result graph controller (232) includes the image controller (270), the text controller (272), and the image graph controller (277) to process the figures from the file (231) that provide evidence for the conclusions of experiments.

The sentence controller (260) is a set of programs that operate to extract the sentences (261) from the file (231). In one embodiment, the sentence controller (260) cleans the text of the file (231) by removing markup language tags, adjusting capitalization, etc. The sentence controller (260) may split a string of text into substrings with each substring being a string that includes a sentence from the original text of the file (231). In one embodiment, the sentence controller (260) may filter the sentences and keep sentences with references to the figures of the file (231).

The sentences (261) are text strings extracted from the file (231). A sentence of the sentences (261) may be stored as a string of text characters. In one embodiment, the sentences (261) are stored in a list that maintains the order of the sentences (261) from the file (231). In one embodiment, the list may be filtered to remove sentences that do not contain a reference to a figure.

The token controller (262) is a set of programs that operate to locate the tokens (263) in the sentences (261). The token controller (262) may identify the start and stop of each token in a sentence.

The tokens (263) identify the boundaries of words in the sentences (261). In one embodiment, a token (of the tokens (263)) may be a substring of a sentence (of the sentences (261)). In one embodiment, a token (of the tokens (263)) may be a set of identifiers that identify the locations of a start character and a stop character in a sentence. Each sentence may include multiple tokens.

The tree controller (264) is a set of programs that operate to generate the trees (265) from the tokens (263) of the sentences (261) of the file (231). In one embodiment, the tree controller (264) uses a neural network (e.g., the Berkeley Neural Parser).

The trees (265) are syntax trees of the sentences (261) to identify the parts of speech of the tokens (263) within the sentences (261). In one embodiment, the trees (265) are graphs with edges identifying parent child relationships between the nodes of a graph. In one embodiment, the nodes of a graph of a tree include a root node, intermediate nodes, and leaf nodes. The leaf nodes correspond to tokens (words, terms, multiword terms, etc.) from a sentence and the intermediate nodes identify parts of speech of the leaf nodes.

The text graph controller (267) is a set of programs that operate to generate the result graphs B (235) from the trees (265). In one embodiment, the text graph controller (267) maps the tokens (263) from the sentences (261) that represent nouns and verbs to nodes of the result graphs B (235). In one embodiment, the text graph controller (267) maps parts of speech identified by the trees (265) to the edges of the result graphs B (235).

In one embodiment, after generating an initial graph (of the result graphs B (235)) for a sentence (of the sentences (261)), the text graph controller (267) processes the graph using the ontology library (252) to identify the entities and corresponding entity types represented by the nodes of the graph. For example, a node of the graph may correspond to the token “BRD9”. The text graph controller (267) identifies the token as an entity defined in the ontology library (252) and identifies the entity type as a protein.

The image controller (270) is a set of programs that operate to extract figures from the file (231) to generate the images (271). The image controller also extracts the figure text (269) that corresponds to the images (271). In one embodiment, the image controller (270) may use rules and logic to identify the images and corresponding image text from the file (231). In one embodiment, the image controller (270) may use machine learning models to identify the images (271) and the figure text (269). For example, the file (231) may be stored in a page friendly format (e.g., a portable document file (PDF)) in which each page of the publication is stored as an image in a file. A machine learning model may identify pages that include figures and the locations of the figures on those pages. The located figures may be extracted as the images (271). Another machine learning model may identify the legend text that corresponds to and describes the figures, which is extracted as the figure text (269).

The images (271) are image files extracted from the file (231). In one embodiment, the file (231) includes the figures as individual image files that the image controller (270) converts to the images (271). In one embodiment, the figures of the file (231) may be contained within larger images, e.g., the image of a page of the file (231). The image controller (270) processes the larger images to extract the figures as the images (271).

The figure text (269) is the text from the file (231) that describes the images (271). Each figure of the file (231) may include legend text that describes the figure. The legend text for one or more figures of the file (231) is extracted as the figure text (269), which corresponds to the images (271).

The text controller (272) is a set of programs that operate to process the images (271) and the figure text (269) to generate the structured text (273). The text controller (272) is further described with FIG. 2B below.

The structured text (273) is strings of nested text with information extracted from the images (271) using the figure text (269). In one embodiment, the structured text (273) includes a JSON formatted string for each image of the images (271). In one embodiment, the structured text (273) identifies the locations of text, panels, and experiment metadata within the images (271). In one embodiment, the structured text (273) includes text that is recognized from the images (271). The structured text (273) may include additional metadata about the images (271). For example, the structured text may identify the types of experiments and the types of techniques used in the experiments that are depicted in the images (271).

The image graph controller (277) is a set of programs that operate to process the structured text (273) to generate one or more of the result graphs B (235). In one embodiment, the image graph controller (277) identifies text that corresponds to entities defined in the ontology library (252) from the structured text (273) and maps the identified text to nodes of the result graphs B (235). In one embodiment, the image graph controller (277) uses the nested structure of the structure text (273) to identify the relationships between the nodes of one or more of the result graphs B (235) and maps the relationships to edges of one or more of the result graphs B (235).

The result graphs B (235) are the graphs generated from the file (231) by the result graph controller (232). The result graphs B (235) include nodes that represent entities defined in the ontology library (252) and include edges that represent relationships between the nodes.

The ontology library (252) defines the entities that may be recognized by the result graph controller (232) from the file (231). The entities defined by the ontology library (252) are input to the token controller (262), the text graph controller (267), and the image graph controller (277), which identify the entities within the text and image extracted from the file (231).

Turning to FIG. 2B, the text controller (272) processes the image (280) and the corresponding legend text (279) to generate the image text (288). The text controller (272) may operate as part of the result graph controller (232) of FIG. 2A.

The image (280) is one of the images (271) from FIG. 2A. The image (280) includes a figure from the file (231) of FIG. 2A.

The legend text (279) is a string from the figure text (269) of FIG. 2A. The legend text (279) is the text from the legend of the figure that corresponds to the image (280).

The text detector (281) is a set of programs that operate to process the image (280) to identify the presence and location of text within the image (280). In one embodiment, the text detector (281) uses machine learning models to identify the presence and location of text. The location may be identified with a bounding box that specifies four points of a rectangle that surrounds text that has been identified in the image (280). The location of the text from the text detector (281) may be input to the text recognizer (282).

The text recognizer (282) is a set of programs that operates to process the image (280) to recognize text within the image (280) and output the text as a string. The text recognizer (282) may process a sub image from the image (280) that corresponds to a bounding box identified by the text detector (281). A machine learning model may then be used to recognize the text from the sub image and output a string of characters that correspond to the text within the sub image.

The panel locator (283) is a set of programs that operates to process the image (280) to identify the location of panels and subpanels within the image (280) or a portion of the image (280). A panel of the image (280) is a portion of the image, which may depict evidence of an experiment. The panels of the image (280) may contain subpanels to further subdivide information contained within the image (280). The image (280) may include multiple panels and subpanels that may identified within the legend text (279). The panel locator (283) may be invoked to identify the location for each panel (or subpanel) identified in the legend text (279). In one embodiment, the panel locator (283) outputs a bit array with each bit corresponding to a pixel from the image (280) and identifying whether the pixel corresponds to a panel.

The experiment detector (284); is a set of programs that operates to process the image (280) to identify metadata about experiments depicted in the image (280). In one embodiment, the experiment detector (284) processes the image (280) with a machine learning model (e.g., a convolutional neural network) that outputs a bounding box and a classification. In one embodiment, the bounding box may be an array of coordinates (e.g., top, left, bottom, right) in the image that identify the location of evidence of an experiment within the image. In one embodiment, the classification may be a categorical value that identifies experiment metadata, which may include the type of evidence, the type of experiment, or technique used in the experiment (e.g., graph, western blot, etc.).

The text generator (285) is a set of programs that operate to process the outputs from the text detector (281), the text recognizer (282), the panel locator (283), and the experiment detector (284) to generate the image text (288). In one embodiment, the text generator (285) creates a nested structure for the image text (288) based on the outputs from the panel locator (283), the experiment detector (284), and the text detector (281). For example, the text generator (285) may include descriptions for the panels, experiment metadata, and text from the image (280) in which the text and description of the experiment metadata may be nested within the description of the panels. Elements for subpanels may be nested within the elements for the panels.

The image text (288) is a portion of the structured text (273) (of FIG. 2A) that corresponds to the image (280). In one embodiment, the image text (288) uses a nested structure to describe the panels, experiment metadata, and text that are identified and located within the image (280).

Turning to FIG. 3, the interface controller (301) is an embodiment of the interface controller (122) of FIG. 1. The interface controller (301) processes information from a request using the project value controller (303), the scoring controller (331), and the summary controller (381) to generate output used for a response.

The project value controller (303) is a collection of programs that identifies a value for a project. The project value controller (303) uses the net present value controller (311) to generate the net present values (317).

The net present value controller (311) generates the net present values (317) from the project data (313) and the accounting data (315) using the target identifiers (305), the disease identifiers (307), and the therapeutic identifiers (309). In one embodiment, for a given project, the net present value controller (311) searches the project data (313) and the accounting data (315) for historical and projected costs and revenue related to the target identifier, the disease identifier, and the therapeutic identifier specified for the project. A discount rate corresponding to the likelihood of success of the project may be applied to the projected costs and revenue to determine the net present value of the project. In one embodiment, the discount rate may be the sum of the cost of capital of an organization and the likelihood that the project will not succeed. For example, the discount rate of 25% may be used when the cost of capital for the organization is 5% with a project that has an 80% likelihood of succeeding (5% +(1-80%)=25%).

The target identifiers (305) are identifiers for targets (such as genes, proteins, pathways, etc.) used by projects defined in the project data (313). The target identifiers (305) may include universally unique identifiers that are mapped to the names of the targets, which may include technical names and common names.

The disease identifiers (307) are identifiers for diseases (e.g., breast cancer) used by projects defined in the project data (313). The disease identifiers (307) may include universally unique identifiers that are mapped to the names of the diseases, which may include technical names and common names.

The therapeutic identifiers (309) are identifiers for therapeutics (e.g., medications) used by projects defined in the project data (313). The therapeutic identifiers (309) may include universally unique identifiers that are mapped to the names of the therapeutics, which may include scientific names and brand names.

The project data (313) is data that specifies a project. The project data (313) includes the identifiers for the targets, diseases, and therapeutics of a project. The project data (313) may include timelines and projections for costs and revenue associated with the project.

The accounting data (315) is data that identifies the historical costs (and revenue) for a project. In one embodiment, the system maintaining the accounting data (315) may not correlate costs directly to projects. The costs attributable to a project may be identified with the net present value controller (311) by identifying costs for the targets, diseases, and therapeutics directly from the accounting data (315). For example, an accounting record may identify a cost for a therapeutic but not the project. The net present value controller may use the therapeutic identifier to correlate the cost for the therapeutic to a specific project.

The net present values (317) are the discounted costs and revenue for the projects identified from the project data (313). In one embodiment, the formula below may be used to calculate the net present value.

where,

$$N P V = {\sum }_{t = 0}^{n} \frac{{C}_{t}}{{( 1 + r )}^{t}}$$

NPV=net present value;
Ct=cash flow (cost or revenue) at time t, which can be positive or negative;
r=discount rate (the required rate of return or hurdle rate);
t=time period (e.g., years or months); and
n=the total number of time periods.

Continuing with FIG. 3, the scoring controller (331) is a collection of programs that identifies the likelihoods of success for projects. The scoring controller (331) uses the biology controller (339), the therapeutic controller (341), the liability controller (343), and the combined score controller (351) to generate the combined score values (353) that may quantify the likelihood of success of the projects being analyzed. For a given project, the scoring controller (331) identifies the target identifiers (305), the disease identifiers (307), the therapeutic identifiers (309), the result graphs (335), and the risk tags (337) that related to the project and are used as inputs to the biology controller (339), the therapeutic controller (341), and the liability controller (343). The result graphs (335) and the risk tags (337) may be subsets of the result graphs B (135) and risk tags B (133) of FIG. 1.

The biology controller (339) is a program that generates the biology score values (345) from the target identifiers (305), the disease identifiers (307), and the result graphs (335). In one embodiment, for a given project, the biology controller (339) searches the result graphs (335) for graphs that include both the target identifier and the disease identifier for the project. In one embodiment, the biology controller (339) scores graphs that indicate that a target is associated with the disease with a “1” and “0” otherwise and averages the scores to generate a biology score value in the range from 0 to 1. In one embodiment, the biology score value may be displayed as a value scaled to a discrete value that is an integer number from 0 to 5.

The therapeutic controller (341) is a program that generates the therapeutic score values (347) from the target identifiers (305), the therapeutic identifiers (309), and the result graphs (335). In one embodiment, for a given project, the therapeutic controller (341) searches the result graphs (335) for graphs that include both the target identifier and the therapeutic identifier for the project. In one embodiment, the therapeutic controller (341) scores graphs that indicate that a target is associated with the therapeutic with a “1” and “0” otherwise and averages the scores to generate a therapeutic score value in the range from 0 to 1. In one embodiment, the therapeutic score value may be displayed as a value scaled to a discrete value that is an integer number from 0 to 5.

The liability controller (343) is a program that generates the liability score values (349) from the target identifiers (305), the disease identifiers (307), the therapeutic identifiers (309), the result graphs (335), and the risk tags (337). In one embodiment, for a given project, the liability controller (343) searches the result graphs (335) for graphs that include the therapeutic identifier for the project. In one embodiment, the liability controller (343) scores graphs with risk tags associated with the therapeutic with a “1” and “0” otherwise and averages the scores to generate a liability score value in the range from 0 to 1. In one embodiment, the liability score value may be displayed as a value scaled to a discrete value that is an integer number from 0 to 5.

The combined score controller (351) is a program that generates the combined score values (353) from the biology score values (345), the therapeutic score values (347), and the liability score values (349). In one embodiment, a biology score value, a therapeutic score value, and a liability score value for a project may be summed to generate the combined score value for the project. In one embodiment, the combined score value is a rational number that includes fractional components from the biology, therapeutic, and liability score values used to generate the combined score value.

Continuing with FIG. 3, the summary controller (381) is a collection of programs that generate text to summarize the result graphs (335). One of the result graphs (335) may be input to the machine learning model (383) to generate one of the summaries (385). In one embodiment, the result graph (e.g., the nodes and edges) is converted to text to form a sentence that is input to the machine learning model (383). In one embodiment, the machine learning model (383) is a large language model that receives the text version of the result graph instructions to generate a summary For example, a result graph may indicate a positive link between a target and a therapeutic and the output from the machine learning model (383) may describe the link in human readable language as a text output forming one of the summaries (385).

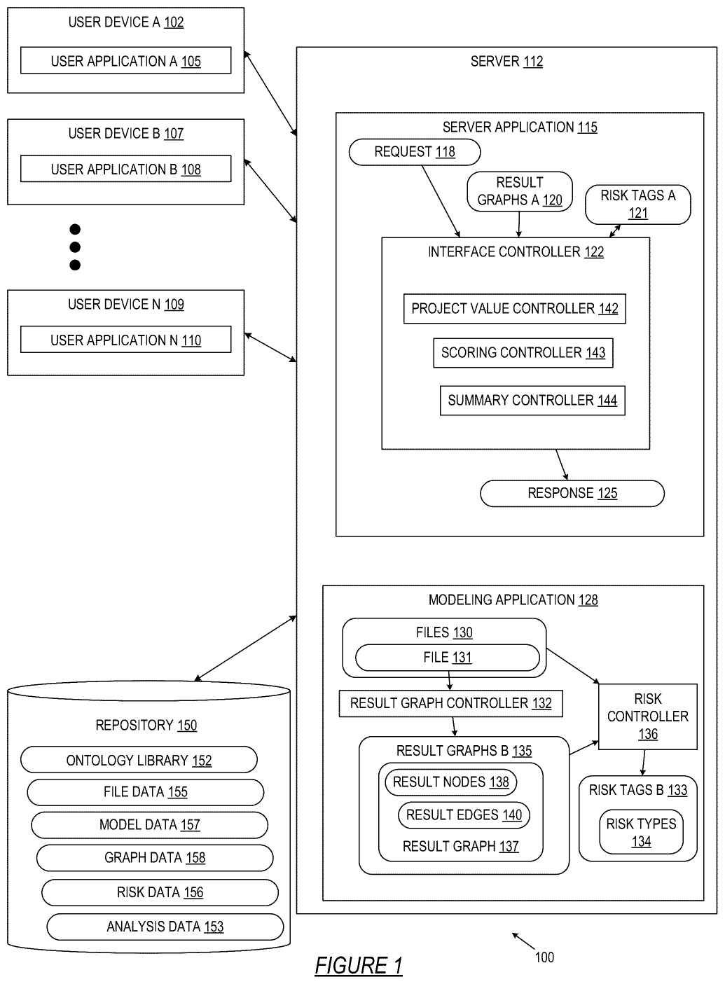

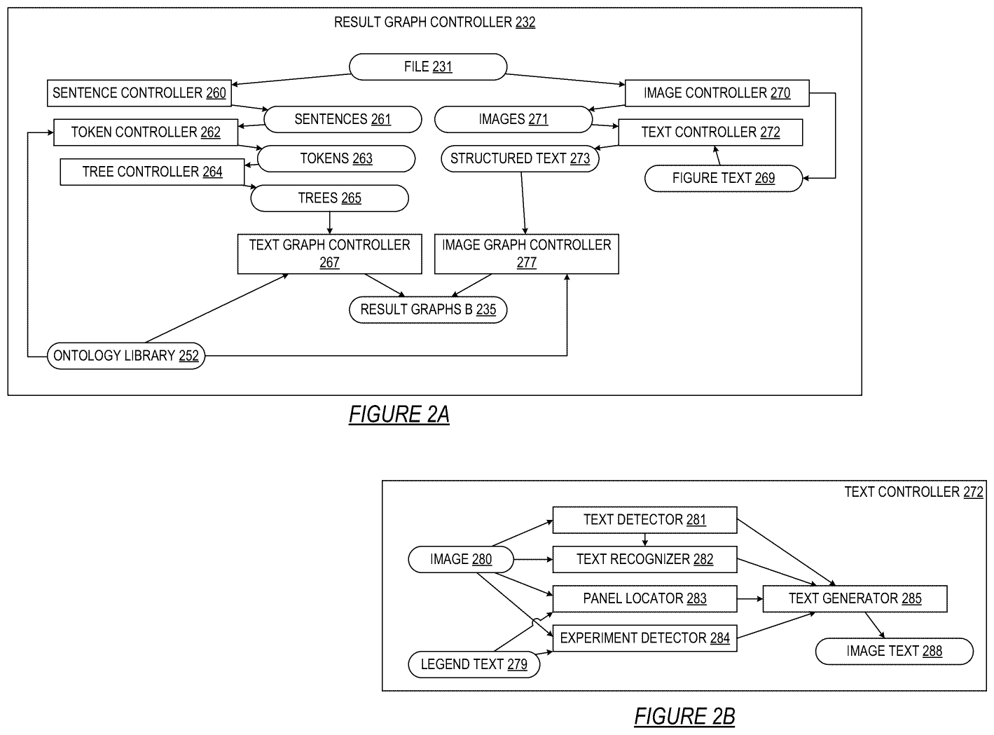

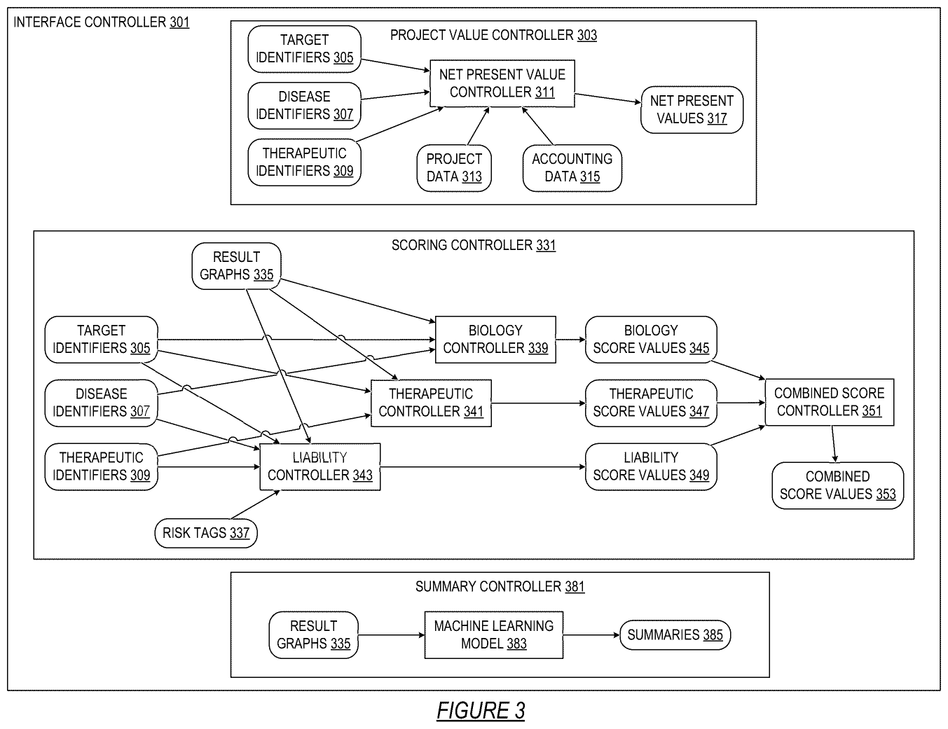

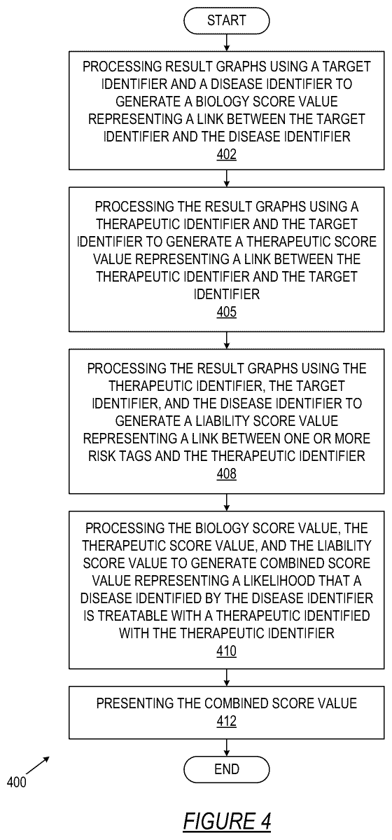

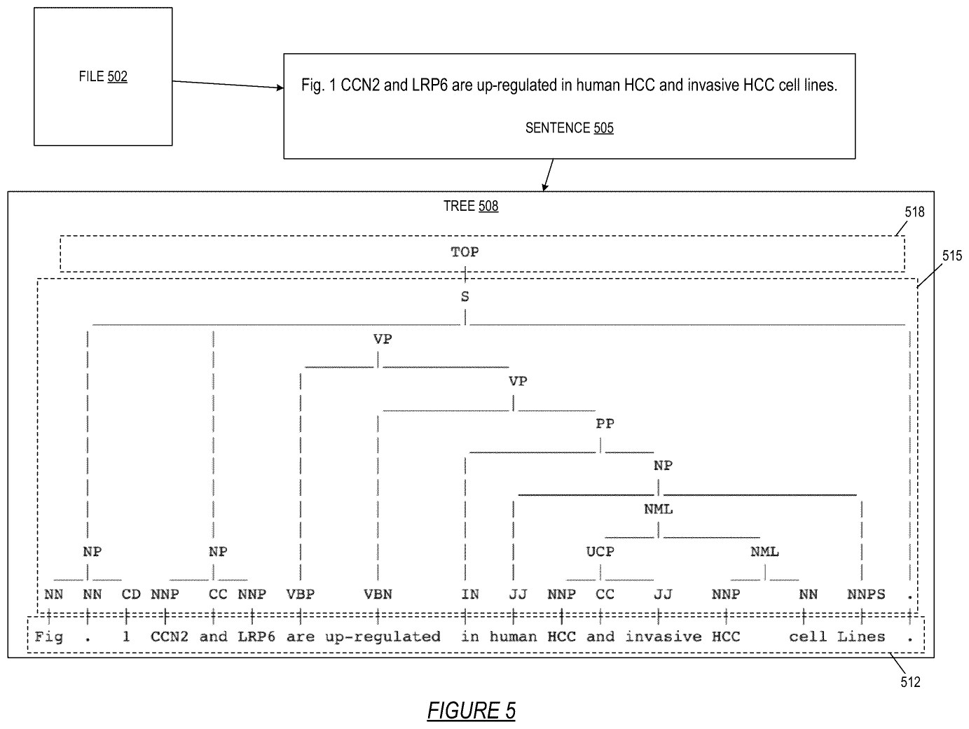

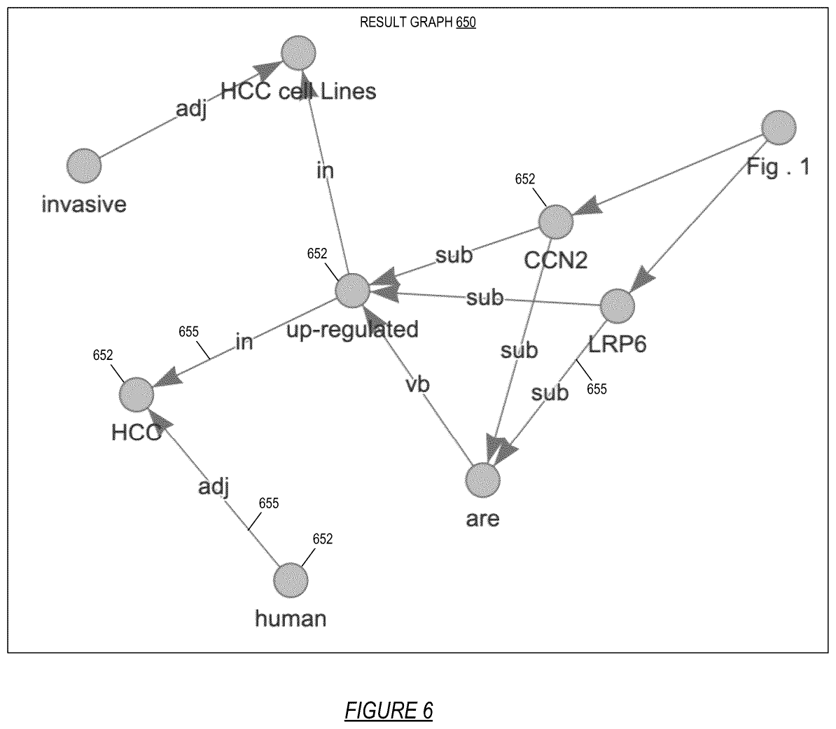

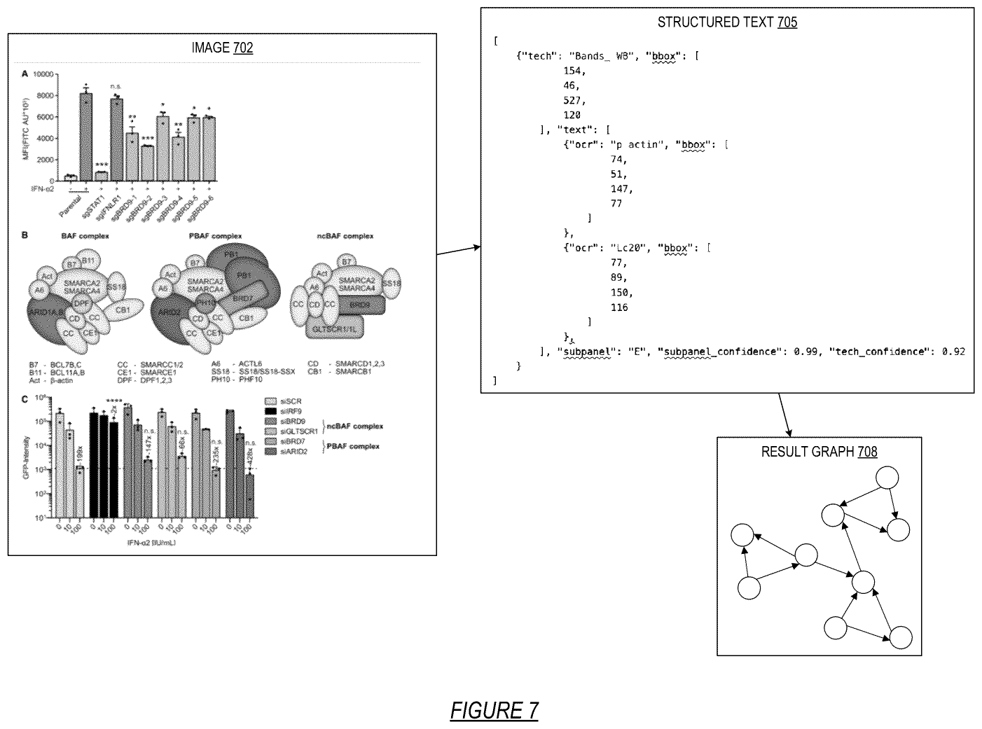

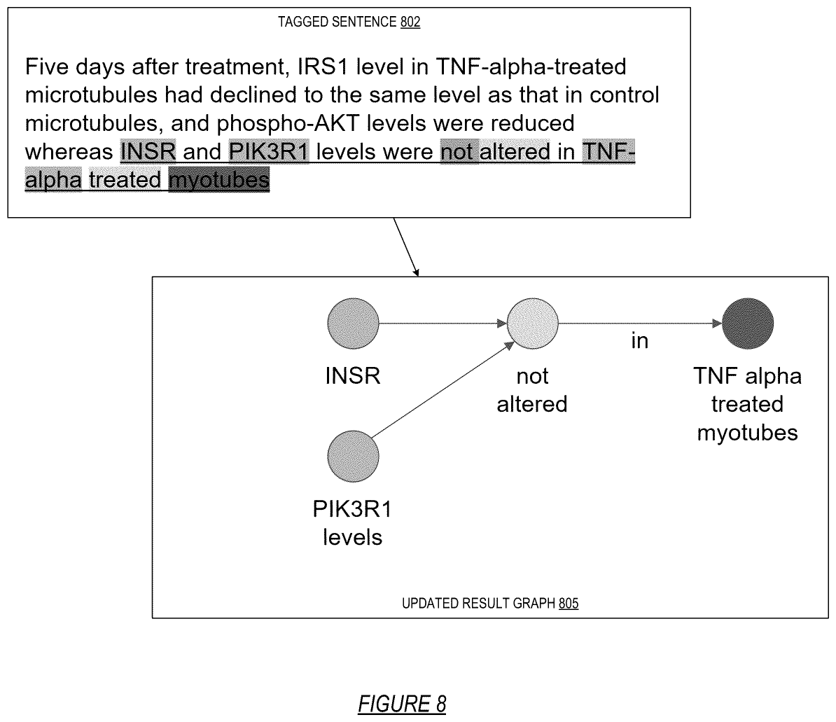

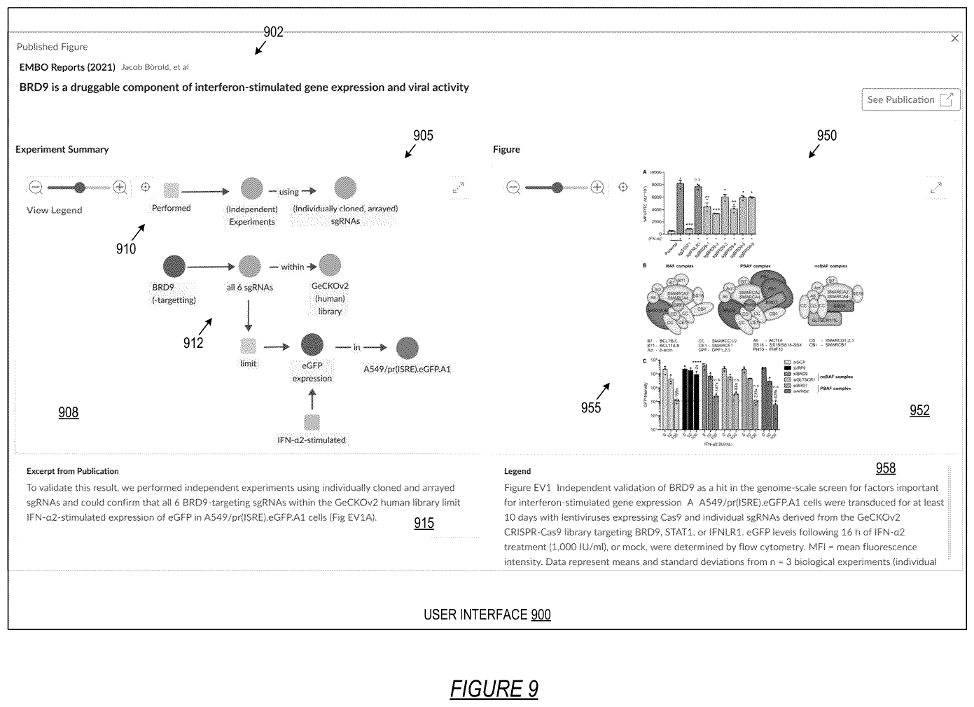

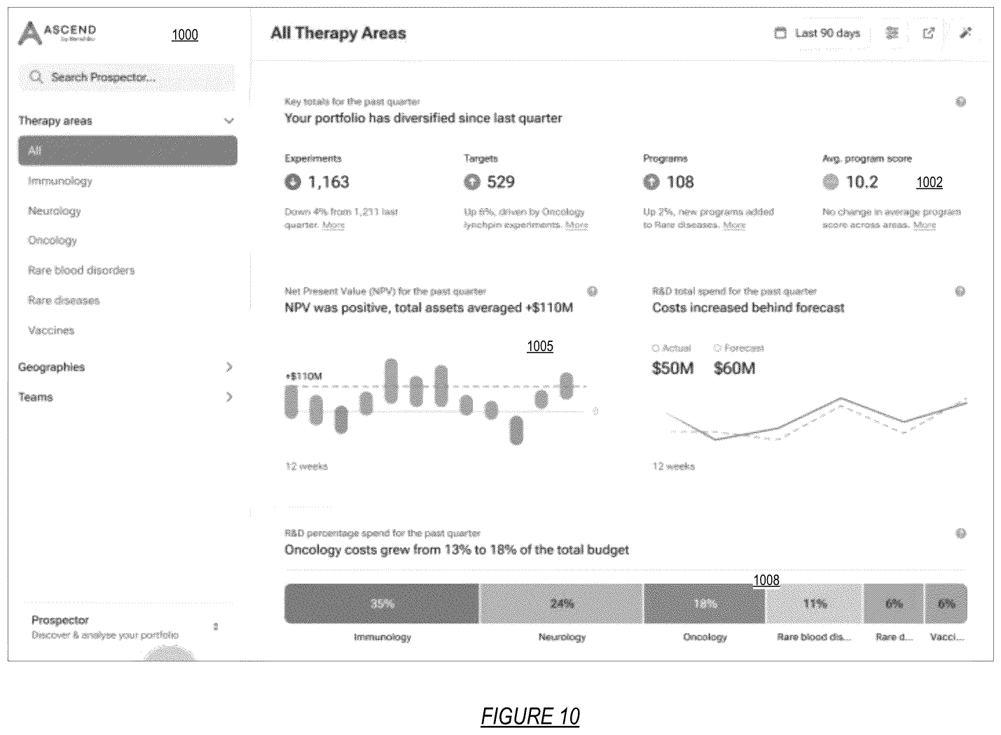

Turning to FIG. 4, the process (400) implements prospecting using biomedical information. The process (400) may be used and implemented by the systems described in the previous figures.

At Step 402, result graphs are processed using a target identifier and a disease identifier to generate a biology score value representing a link between the target identifier and the disease identifier. In one embodiment, generating the biology score value includes searching the result graphs for a set of target result graphs that include the target identifier and the disease identifier. Generating the biology score value may further include processing the set of target result graphs to generate the biology score value representing a likelihood that a target represented by the target identifier affects the disease represented by the disease identifier. In one embodiment, a value of “1” may be assigned for each result graph including a positive association and a value of “0” may be assigned for each negative association. In one embodiment, a machine learning model may receive the result graph as an input and output a value from “0” to “1” with higher values (e.g., from “0.7” to “1”) identified as promoters, lower values (e.g., from “0” to less than “0.5”) identified as demoters, and values in between (between “0.5” and less than “0.7”) as passive. The assigned values may then be averaged to generate the biology score value in which a higher value corresponds to a higher likelihood of success for the project.

In one embodiment, a minimum threshold (e.g., 3, 4, 5, etc.) for the number of different sources of evidence may be used for determining the biology score value. A source of evidence may include publications of experiments from which a result graph has been generated. As an example, the biology score value may not be determined when the system is unable to identify the result graphs from three different publications that relate to the biology (i.e., the target to the disease).

In one embodiment, the result graphs are generated by processing multiple files with biomedical information. The result graphs include a result graph that includes a node that corresponds to text from a file describing an experiment related to one or more of the target identifier, the disease identifier, the therapeutic identifier.

At Step 405, the result graphs are processed using a therapeutic identifier and the target identifier to generate a therapeutic score value representing a link between the therapeutic identifier and the target identifier. In one embodiment, generating the therapeutic score value includes searching the result graphs for a set of therapeutic result graphs each including the therapeutic identifier and the target identifier. Generating the therapeutic score value may further include processing the set of therapeutic result graphs to generate the therapeutic score value representing a likelihood that the therapeutic represented by the therapeutic identifier affects a target represented by the target identifier. In one embodiment, a value of “1” may be assigned for each result graph including a positive association and a value of “0” may be assigned for each negative association. In one embodiment, a machine learning model may receive the result graph as an input and output a value from “0” to “1” with higher values (e.g., from “0.7” to “1”) identified as promoters, lower values (e.g., from “0” to less than “0.5”) identified as demoters, and values in between (between “0.5” and less than “0.7”) as passive. The assigned values may then be averaged to generate the therapeutic score value in which a higher value corresponds to a higher likelihood of success for the project.

In one embodiment, a minimum threshold (e.g., 3, 4, 5, etc.) for the number of different sources of evidence may be used for determining the therapeutic value score. A source of evidence may include publications of experiments from which a result graph has been generated. As an example, the therapeutic score value may not be determined when the system is unable to identify the result graphs from three different publications that relate the therapeutic with the target.

At Step 408, the result graphs are processed using the therapeutic identifier, the target identifier, and the disease identifier to generate a liability score value representing a link between one or more risk tags and the therapeutic identifier. In one embodiment, generating the liability score value includes searching the result graphs for a set of liability result graphs linked to a set of risk tags identifying a set of risk types for a set of experiments involving the therapeutic identifier. Generating the liability score value further includes processing the set of liability result graphs and the set of risk tags to generate the liability score value representing a likelihood that the therapeutic represented by the therapeutic identifier is associated with an adverse event. In one embodiment, a value of “1” may be assigned for each result graph that includes the therapeutic identifier but does not include a risk tag and a value of “0” may be assigned for each result graph that includes the therapeutic identifier and does include a risk tag. In one embodiment, a machine learning model may receive the result graph as an input and output a value from “0” to “1” with higher values (e.g., from “0.7” to “1”) identified as promoters, lower values (e.g., from “0” to less than “0.5”) identified as demoters, and values in between (between “0.5” and less than “0.7”) as passive. The assigned values may then be averaged to generate the liability score value in which a higher value means lower risk of liability and a higher likelihood of success for the project.

In one embodiment, a minimum threshold (e.g., 3, 4, 5, etc.) for the number of different sources of evidence may be used for determining the liability score value. A source of evidence may include publications of experiments from which a result graph has been generated. As an example, the liability score value may not be determined when the system is unable to identify the result graphs from three different publications that relate two or more of the target, the therapeutic, and the disease.

At Step 410, the biology score value, the therapeutic score value, and the liability score value are processed to generate combined score value. The combined score value may represent a likelihood that a disease identified by the disease identifier is treatable with a therapeutic identified with the therapeutic identifier. In one embodiment, the combined score value may represent the likelihood of success of a project.

At Step 412, the combined score value is presented. The combined score value may be displayed on a user device.

In one embodiment, a summary may be presented. Generating the summary may include identifying a result graph that affects the biology score value, the therapeutic score value, or the liability score value. For example, a recently published paper (e.g., within the last 7 days) may have increased or decreased the biology score value, the therapeutic score value, and the liability score value.

The summary an experiment represented by the result graph may be generated by processing the result graph using a language model. In one embodiment, the nodes and edges of the result graph nodes may be converted to text to form a pseudo sentence that may be converted to tokens or vectors that are input to a large language model that outputs the summary.

The summary may be identified and presented as a promoter summary when the result graph increases one or more of the biology score value, the therapeutic score value, and the liability score value. The summary may be identified and presented as a demoter summary when the result graph decreases one or more of the biology score value, the therapeutic score value, and the liability score value. The summary may be identified and presented as a passive summary when the result graph is associated with a risk tag but the risk tag in the result graph is not associated with the therapeutic of the project. For example, a paper that mentions multiple therapeutics may identify a risk for one of the therapeutics of the paper that is different from the therapeutic used by the project.

In one embodiment, a net present value of the project may be presented. The net present value may be generated by processing project data and accounting data using the target identifier, the disease identifier, and the therapeutic identifier to generate the net present value for a project.

In one embodiment, a timeline with the combined score value may be presented. The timeline may be generated by processing multiple combined score values for different dates to generate a timeline of combined score values.

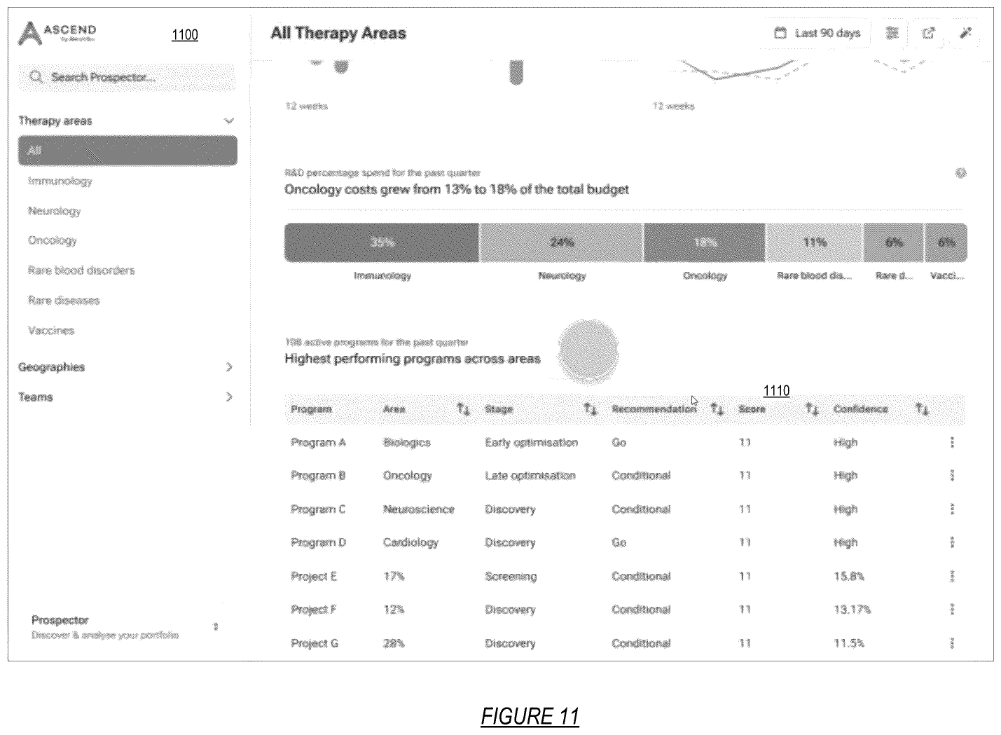

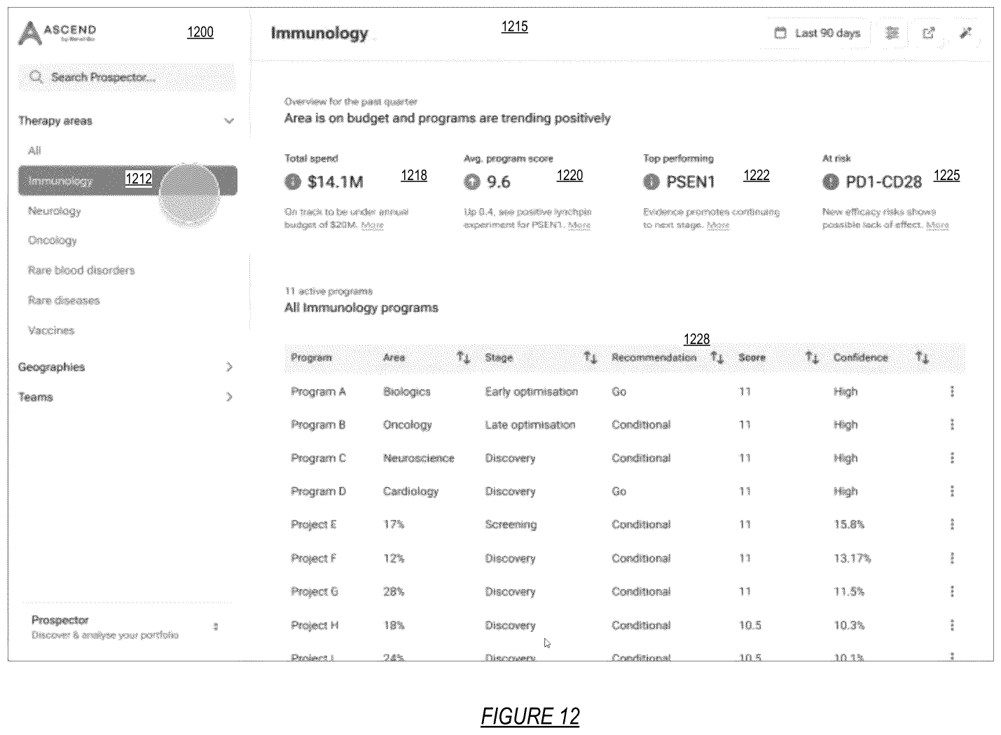

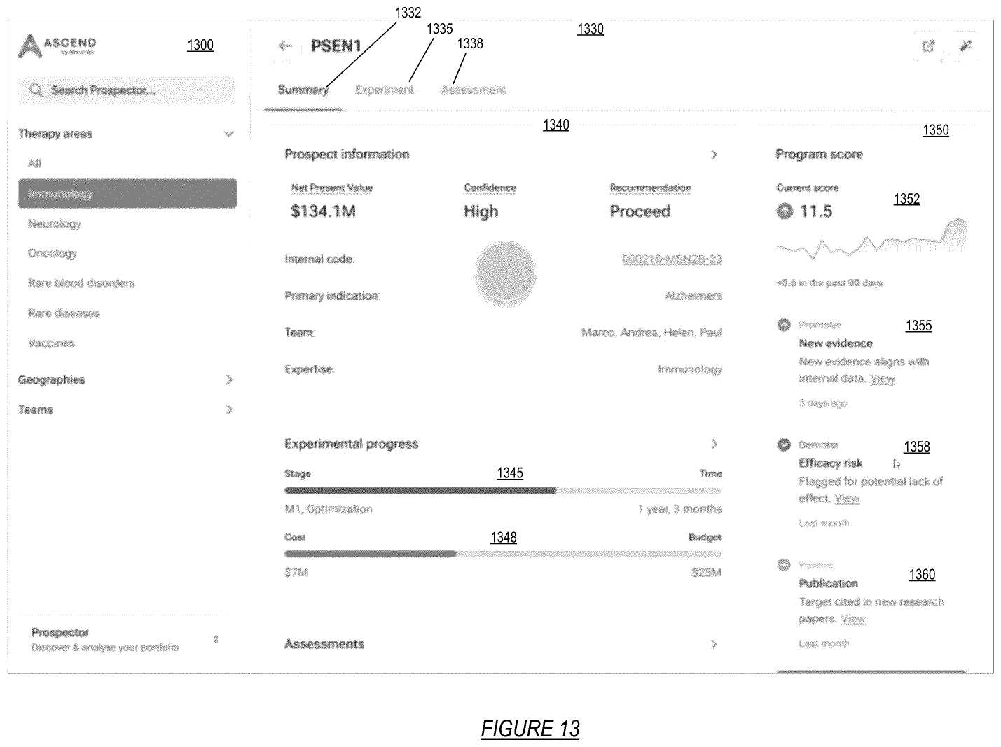

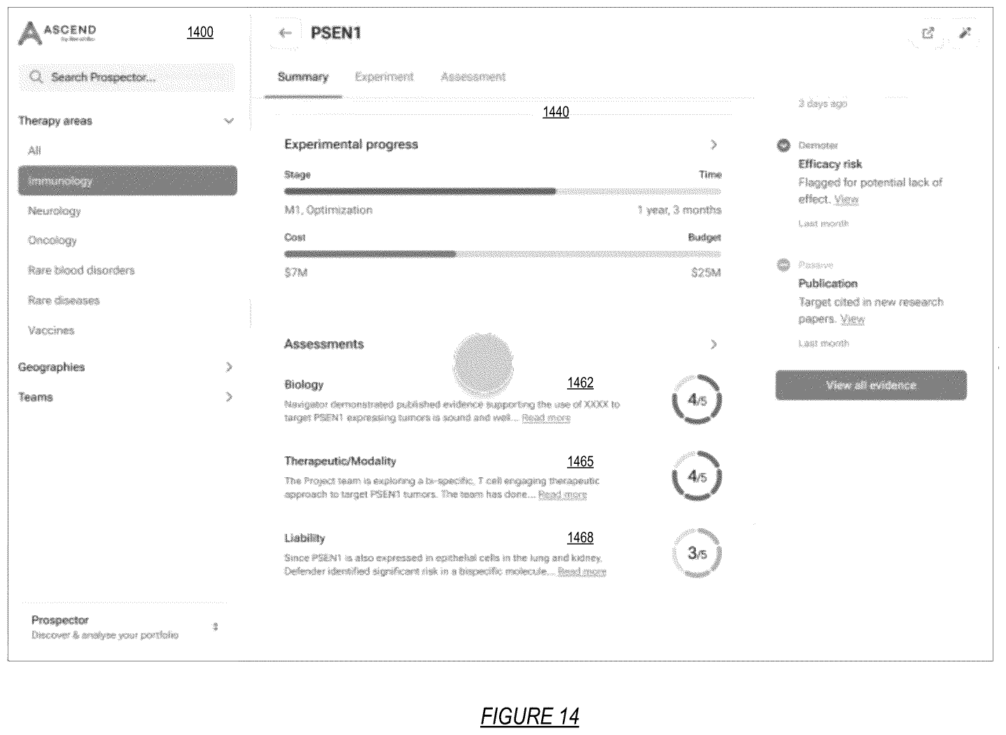

Turning to FIG. 5, the file (502) is shown from which the sentence (505) is extracted, which is used to generate the tree (508), which is used to generate the result graph (650) (of FIG. 6). The file (502), the sentence (505), the tree (508), and the result graph (650) (of FIG. 6) may be stored as electronic files, transmitted in messages, and displayed on a user interface.

The file (502) is a collection of biomedical information, which may include, but is not limited to, a writing of biomedical literature with sentences and figures stored as text and images. Different sources of biomedical information may be used. The file (502) is processed to extract the sentence (505).

The sentence (505) is a sentence from the file (502). The sentence (505) is stored as a string of characters. In one embodiment, the sentence (505) is tokenized to identify the locations of entities within the sentence (505). For example, the entities recognized from the sentence (505) may include “CCN2”, “LRP6”, “HCC”, and “HCC cell lines”. The sentence (505) is processed to generate the tree (508).

The tree (508) is a data structure that identifies semantic relationships of the words of the sentence (505). The tree (508) includes the leaf nodes (512), the intermediate nodes (515), and the root node (518).

The leaf nodes (512) correspond to the words from the sentence (505). The leaf nodes have no child nodes. The leaf nodes have parent nodes in the intermediate nodes (515).

The intermediate nodes (515) include values that identify the parts of speech of the leaf nodes (512). The intermediate nodes (515) having leaf nodes as direct children nodes identify the parts of speech of the words represented by the leaf nodes. The intermediate nodes (515) that do not have leaf nodes as direct children nodes identify the parts of speech of groups of one or more words, i.e., phrases, of the sentence (505).

The root node (518) is the top of the tree (508). The root node (518) has no parent node.

Turning to FIG. 6, the result graph (650) is a data structure that represents the sentence (505) (of FIG. 5). The result graph (650) may be generated from the sentence (505) and the tree (508) (of FIG. 5). The nodes of the result graph (650) represent nouns (e.g., “CCN2”, “HCC”, etc.) and verbs (e.g., “up-regulated”, “are”, etc.) from the sentence (505) (of FIG. 5). The edges (655) identify semantic relationships (e.g., subject “sub”, verb “vb”, adjective “adj”) between the words of the nodes (652) of the sentence (505) (of FIG. 5). The result graph (650) is a directed acyclic graph.

Turning to FIG. 7, the image (702) is shown from which the structured text (705) is generated, which is used to generate the result graph (708). The image (702), the structured text (705), and the result graph (708) may be stored as electronic files, transmitted in messages, and displayed on a user interface.

The image (702) is a figure from a file (e.g., the file (502) of FIG. 5, which may be from a biomedical publication). In one embodiment, the image (702) is an image file that is included with or as part of the file (502) of FIG. 5. In one embodiment, the image (702) is extracted from an image of a page of a publication stored as the file (502) of FIG. 5. The image (702) includes three panels labeled “A”, “B”, and “C”. The “B” panel includes three subpanels labeled “BAF complex”, “PBAF complex”, and “ncBAF complex”. The image (702) is processed to recognize the locations of the panels, subpanels, and text using machine learning models. After being located, the text from the image is recognized and stored as text (i.e., strings of characters). The panel, subpanel, and text locations along with the recognized text are processed to generate the structured text (705).

The structured text (705) is a string of text characters that represents the image (702). In one embodiment, the structured text (705) includes nested lists that form a hierarchical structure patterned after the hierarchical structure of the panels, subpanels, and text from the image (702). The structured text (705) is processed to generate the result graph (708).

The result graph (708) is a data structure that represents the figure, corresponding to the image (702), from a file (e.g., the file (502) of FIG. 5). The result graph (708) includes nodes and edges. The nodes represent nouns and verbs identified in the structured text (705). The edges may represent the nested relationships between the panels, subpanels, and text of the image (702) described in the structured text (705).

Turning to FIG. 8, the tagged sentence (802) is generated from a sentence and used to generate the updated result graph (805). The tagged sentence (802) and the updated result graph (805) may be stored as electronic files, transmitted in messages, and displayed on a user interface.

The tagged sentence (802) is a sentence from a file that has been processed to generate the updated result graph (805). The sentence from which the tagged sentence is derived is input to a model to tag the entities in the sentence to generate the tagged sentence (802). The model may be a rules-based model, an artificial intelligence model, combinations thereof, etc.

As an example, the underlined portion (“INSR and PIK3R1 levels were not altered in TNF-alpha treated myotubes”) is tagged by the model. The terms “INSR”, “PIK3R1”, and “TNF-alpha” may be tagged as one type of entity that is presented as green when displayed on a user interface. The term “not” is tagged and may be displayed as orange. The terms “altered” and “treated” are tagged and may be displayed as pink. The term “myotubes” is tagged and may be displayed as red. After being identified in the sentence, the tags may be applied to the graph to generate the updated result graph (805).

The updated result graph (805) is an updated version of a graph of the sentence used to generate the tagged sentence (802). The graph is updated to label the nodes of the graph with the tags from the tagged sentence. For example, the nodes corresponding to “INSR” and “PIK3R1” are labeled with tags identified in the tagged sentence and may be displayed as green. The node corresponding to “altered” is tagged and displayed as pink. The node corresponding to “myotubes” is tagged and displayed as red.

Turning to FIG. 9, the user interface (900) displays information from a file, which may be a publication of biomedical literature. Different sources of files may be used. The user interface (900) may display the information on a user device after receiving a response to a request for the information transmitted to a server application. For example, the request may be for a publication that includes evidence linking the proteins “BRD9” and “A549”. The user interface displays the header section (902), the summary section (905), and the figure section (950).

The header section (902) includes text identifying the file being displayed. In one embodiment, the text in the header section (902) includes the name of the publication, the name of the author, the title of the publication, etc., which may be extracted from the file. Additional sources of information may be used, including patents, ELN data, summary documents, portfolio documents, scientific data in raw/table form, presentations, etc., and similar information may be extracted.

The summary section (905) displays information from the text of the file identified in the header section (902). The summary section (905) includes the graph section (908) and the excerpt section (915).

The graph section (908) includes the result graphs (910) and (912). The result graphs (910) and (912) were generated from the sentence displayed in the excerpt section (915). The result graph (912) shows the link between the proteins “BRD9” and “A549”, which conforms to the request that prompted the response with the information displayed in the user interface (900).

The excerpt section (915) displays a sentence from the file identified in the header section (902). The sentence in the excerpt section (915) is the basis from which the result graphs (910) and (912) were generated by tokenizing the sentence, generating a tree from the tokens, and generating the result graphs (910) and (912) from the tokens and tree.

The figure section (950) displays information from the figures of the file identified in the header section (902). The figure section (950) includes the image section (952) and the legend section (958).

The image section (952) displays the image (955). The image (955) was extracted from the file identified in the header section (902). The image (955) corresponds to the text from the legend section (958). The image (955) corresponds to the result graph (912) because the sentence shown in the excerpt section (915) identifies the figure (“Fig EV1A”) that corresponds to the image (955).

The legend section (958) displays the text of the legend that corresponds to the figure of the image (955). In one embodiment, the text of the legend section (955) may be processed to generate one or more graphs from the sentence in the legend section (958).

Turning to FIG. 10, the user interface (1000) displays a dashboard of information aggregated from multiple projects of an organization. Each project may be researching the efficacy of a therapeutic (e.g., a medication or treatment) for a disease based on a target (such as a gene, a protein, a pathway, and antibody, etc.).

The interface element (1002) includes a score, which may be an average score generated by averaging the scores for each of the projects of the organization. In one embodiment, the averaging used to generate the score may be weighted average using the net present values of the projects to weight the scores of the projects.

The interface element (1005) includes a timeline of the net present values for the projects of the organization. For each point in the timeline, the net present values of the different projects may be summed to form the value used in the timeline.

The interface element (1008) includes a chart with a single stacked bar. The elements of the stacked bar each correspond to a different group of projects within the organization and identify the relative cost as a percentage of the whole costs for all of the projects combined.

Turning to FIG. 11, the user interface (1100) shows the user interface (1000) of FIG. 10 after scrolling down to reveal the interface element (1110). The interface element (1110) includes a table with a row for each project of the organization. The table of the interface element (1110) may be filtered or sorted by different therapy areas. The table of the interface element (1110) includes several columns of information for the projects of the organization. One of the columns is for the score of a project. A score may be the combined score generated from a biology score value, a therapeutic score value, and a liability score value. In one embodiment, additional columns may be included to display each of the biology score values, therapeutic score values, and liability score values for the different projects.

Turning to FIG. 12, the user interface (1200) is updated from the user interface (1100) of FIG. 11 after selecting the interface element (1212). Selecting the interface element (1212) updates the view (1215) to show information about a group of projects for a therapy area, e.g., “immunology”. The interface element (1218) displays a value indicating the total costs for the group of projects. The interface element (1220) displays and average score for the group of projects (which may be weighted by the net present value of the projects within the group). The interface element (1222) identifies the therapeutic of the project with the highest net present value within the group of projects. The interface element (1225) identifies the therapeutic of the project for which the liability score value has recently decreased (indicating a lower likelihood of success for the project). The interface element (1228) includes a table that may be filtered to show the projects that are part of the group.

Turning to FIG. 13, user interface (1300) is updated from the user interface (1200) of FIG. 12 to show the view (1330). Display of the view (1330) may be in response to selecting a row from the table of the interface element (1228) of FIG. 12 or from selecting the interface element (1222) FIG. 12. The view (1330) includes several interface elements to display information about a project. The view includes the interface elements (1332), (1335), and (1338) to select between different views for the project. Selection of the interface element (1332) is for the summary view (1340).

The summary view (1340) displays information about project including the net present value of the project and the team members associated with the project. Additionally the experimental progress is shown with a milestone timeline (1345) above a cost line chart (1348). The milestone timeline (1345) identifies that amount of time projected to remain for the project and provides an indication of the overall time frame for the project. The cost line chart (1348) identifies the budget for the project and the amount of costs already incurred for the project.

The view (1330) also includes the interface element (1350) with additional information about the project. The interface element (1350) includes the score timeline (1352) that shows the current and historical scores (e.g., the current and historical combined scores) for the project. The interface element (1350) also includes the interface elements (1355), (1358), and (1360).

The interface element (1355) includes a summary generated from a result graph identified as a “promoter” that increased the combined score of the project when analyzed by the system. The interface element (1358) includes a summary generated from a different result graph identified as a “demoter” that decreased the combined score of the project when analyzed by the system. The interface element (1360) includes a summary generated from another result graph identified as “passive” that did not affect the combined score of the project.

Turning to FIG. 14, the user interface (1400) shows the user interface (1300) of FIG. 13 after scrolling down. The summary view (1440) includes the interface elements (1462), (1465), and (1468).

The interface element (1462) provides a biology assessment to show support for a target to affect a disease. The biology assessment may show that targets are expressed in relevant tissues, cell types, and species. The biology assessment may further show strengths of scientific evidence linking a target to an indication, which may include direct or indirect linkages. The biology assessment may further show resolution of the molecular mechanism of a target and involvement in disease pathogenesis. The biology assessment includes a biology score value, which may analyze conflicting evidence and be aligned with internal experimental data when available. The interface element (1462) may display the score value as a discrete integer with summary text that summarizes the result graphs used to generate the biology score value.

The interface element (1465) provides an of evidence to support therapeutic or modality appropriateness and feasibility, which may also be referred to as a therapeutic assessment. The therapeutic assessment may show supporting evidence that illustrates the biology of the therapeutic/modality/intervention. The therapeutic assessment may further show precedence for similar modalities in relevant indications. The therapeutic assessment may further identify proposed modality approaches to yield high on target specificity and efficacy. The therapeutic assessment includes a therapeutic score value that may incorporate modality mitigation strategies to increase on target specificity and efficacy and be aligned with internal experimental data when available. The interface element (1465) displays a therapeutic score value as a discrete integer with summary text that summarizes the result graphs used to generate the therapeutic score value.

The interface element (1468) provides a liability assessment to show safety risks in the evidence that may impact clinical translation. The liability assessment may show indications of potential safety or efficacy risks, which may be pre-clinical in vitro or inferred expression level insights. The liability assessment may show adverse event or contraindications that relate to the Target in in vivo models. The liability assessment may show adverse Events or contraindications reported in human clinical data (e.g., from the Food and Drug Administration (FDA)). The liability assessment includes a liability score value that may incorporate experimental or patient stratification mitigation strategies and be aligned with internal experimental data when available. The interface element (1468) displays a liability score value as a discreet integer with summary text that summarizes the result graphs used to generate the liability score value.

Turning to FIG. 15, the user interface (1500) may be displayed after selection of the interface element (1335) of FIG. 13. The user interface (1500) displays the experiment view (1547) with information about the experiment being conducted for the project to study the efficacy of using the therapeutic to treat the disease by way of the target. The user interface (1500) includes, within the experiment view (1547), the milestone timeline (1545) above the cost line chart (1548). The user interface (1500) further includes the interface element (1570), which provides text describing an objective of the experiment with a link.

Turning to FIG. 16, the user interface (1600) may be displayed after selection of the link from the interface element (1570) of FIG. 15. The interface element (1600) includes the graph (1672) and the text (1675). The graph (1672) is similar to a result graph and shows relationships between the target, the therapeutic, and the disease being studied in the experiment for the project. The text (1675) describes the objective of the experiment and may be generated using a machine learning model from the graph (1672).

Turning to FIG. 17, the user interface (1700) shows the user interface (1500) of FIG. 15 after scrolling down. The experiment view (1747) displays molecular details related to the experiment. The molecular details displayed include information about targets, methods of action, compounds, evidence depth, indications, and the biomarkers.

Turning to FIG. 18, the user interface (1800) shows the user interface (1700) of FIG. 17 after scrolling further down. The experiment view (1847) displays details related to the design of the experiment. The experiment details displayed include information about approaches, conditions, molecular markers, and techniques.

Turning to FIG. 19, the user interface (1900) may be displayed after selection of the interface element (1338) of FIG. 13. The assessment view (1978) and the score view (1980) of the user interface element (1900) displays additional information about the experiment with information about the individual scores that make up the combined score for the experiment.

The assessment view (1978) includes the biology interface element (1982). The biology interface element (1982) includes summary text that summarizes the findings from the biomedical information that is converted into result graphs and used to generate the biology score value for the experiment. Below the summary text are interface elements that may be used to view additional information from a source of biomedical information. For example, the interface element (1985) may display one or more of an image from a source of biomedical information (e.g., a paper discussing an experiment that used a therapeutic), such as the image (955) of FIG. 9, or a result graph, such as the result graphs (910) and (912) of FIG. 9. In one embodiment, selecting the interface element (1985) may display the user interface (900) of FIG. 9.

The score view (1980) displays the biology score value, therapeutic score value, and the liability score value with annotations to identify when the values were updated. The values are shown numerically and with a circular bar around the value that may be color coded to the value. A score of one may be coded as red, scores of two or three may be coded as yellow, scores of four or five may be coded as green.

Turning to FIG. 20, the analysis view (2088) of the user interface (2000) is shown as an alternative to the assessment view (1978) of FIG. 19. The analysis view (2088) includes the biology, therapeutic, liability score values displayed numerically and with a set of bars to the left of the number for the value. The bars may be color coded with red for scores of one or two, yellow for a score of three, or green for scores of four or five. Different codings and colors may be used.

The analysis view (2088) may include the overview section (2090) and the biology section (2092) as well as therapeutic and liability sections (not shown). The overview section (2090) includes recommendation text that is about a sentence long generated by a large language model in response to a prompt that includes the biology, therapeutic, liability score values for the experiment. The biology section (2092) includes a summary sentence describing the link between the target and the disease and a summary paragraph that provides additional information gathered from the result graphs used to generate the biology score value. The summary sentence and paragraph may be generated using a large language model with a prompt that includes the target, the disease, and text versions of one or more of the result graphs used to generate the biology score value.

Turning to FIG. 21, the user interface (2100) shows the user interface (1900) of FIG. 19 after scrolling down to display the therapeutic interface element (2195). The therapeutic interface element (2195) includes summary text that summarizes the findings from the biomedical information that is converted into result graphs and used to generate the therapeutic score value for the experiment. Below the summary text are interface elements that may be used to view additional information from a source of biomedical information, including evidence images and result graphs as described above with the biology interface element (1982) of FIG. 19.

Turning to FIG. 22, the user interface (2200) shows the user interface (2100) of FIG. 21 after scrolling down to display the liability interface element (2298). The interface element (2298) includes summary text that summarizes the findings from the biomedical information that is converted into result graphs and used to generate the liability score value for the experiment. Below the summary text are interface elements that may be used to view additional information from a source of biomedical information, including evidence images and result graphs as described above with the biology interface element (1982) of FIG. 19.

Embodiments of the invention may be implemented on a computing system. Any combination of a mobile, a desktop, a server, a router, a switch, an embedded device, or other types of hardware may be used. For example, as shown in FIG. 23A, the computing system (2300) may include one or more computer processor(s) (2302), non-persistent storage (2304) (e.g., volatile memory, such as a random access memory (RAM), cache memory), persistent storage (2306) (e.g., a hard disk, an optical drive such as a compact disk (CD) drive or a digital versatile disk (DVD) drive, a flash memory, etc.), a communication interface (2312) (e.g., Bluetooth interface, infrared interface, network interface, optical interface, etc.), and numerous other elements and functionalities.

The computer processor(s) (2302) may be an integrated circuit for processing instructions. For example, the computer processor(s) (2302) may be one or more cores or micro-cores of a processor. The computing system (2300) may also include one or more input device(s) (2310), such as a touchscreen, a keyboard, a mouse, a microphone, a touchpad, an electronic pen, or any other type of input device.

The communication interface (2312) may include an integrated circuit for connecting the computing system (2300) to a network (not shown) (e.g., a local area network (LAN), a wide area network (WAN) such as the Internet, a mobile network, or any other type of network) and/or to another device, such as another computing device.

Further, the computing system (2300) may include one or more output device(s) (2308), such as a screen (e.g., a liquid crystal display (LCD), a plasma display, a touchscreen, a cathode ray tube (CRT) monitor, a projector, or other display device), a printer, an external storage, or any other output device. One or more of the output device(s) (2308) may be the same or different from the input device(s) (2310). The input and output device(s) (2310 and (2308)) may be locally or remotely connected to the computer processor(s) (2302), non-persistent storage (2304), and persistent storage (2306). Many different types of computing systems exist, and the aforementioned input and output device(s) (2310 and (2308)) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (2300) in FIG. 23A may be connected to or be a part of a network. For example, as shown in FIG. 23B, the network (2320) may include multiple nodes (e.g., node X (2322), node Y (2324)). Each node may correspond to a computing system, such as the computing system (2300) shown in FIG. 23A, or a group of nodes combined may correspond to the computing system (2300) shown in FIG. 23A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (2300) may be located at a remote location and connected to the other elements over a network.

Although not shown in FIG. 23B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (2322), node Y (2324)) in the network (2320) may be configured to provide services for a client device (2326). For example, the nodes may be part of a cloud computing system. The nodes may include functionality to receive requests from the client device (2326) and transmit responses to the client device (2326). The client device (2326) may be a computing system, such as the computing system (2300) shown in FIG. 23A. Further, the client device (2326) may include and/or perform all or a portion of one or more embodiments of the invention.

The computing system (2300) or group of computing systems described in FIGS. 23A and 23B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel endpoints enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (2300) in FIG. 23A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (2300) of FIG. 23A, while performing one or more embodiments of the invention, may perform data comparison. Data comparison may be used to compare two or more data values (e.g., A, B). For example, one or more embodiments may determine whether A>B, A=B, A!=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (2300) in FIG. 23A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (2300) of FIG. 23A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (2300) of FIG. 23A and the nodes (e.g., node X (2322), node Y (2324)) and/ or client device (2326) in FIG. 23B. Other functions may be performed using one or more embodiments of the invention.

As used herein, the term “connected to” contemplates multiple meanings. A connection may be direct or indirect (e.g., through another component or network). A connection may be wired or wireless. A connection may be temporary, permanent, or semi-permanent communication channel between two entities.

The various descriptions of the figures may be combined and may include or be included within the features described in the other figures of the application. The various elements, systems, components, and steps shown in the figures may be omitted, repeated, combined, and/or altered as shown from the figures. Accordingly, the scope of the present disclosure should not be considered limited to the specific arrangements shown in the figures.

In the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Further, unless expressly stated otherwise, the word “or” is an “inclusive or” and, as such includes “and.” Further, items joined by an or may include any combination of the items with any number of each item unless expressly stated otherwise.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
processing a plurality of result graphs using a target identifier and a disease identifier to generate a biology score value representing a link between the target identifier and the disease identifier;
processing the plurality of result graphs using a therapeutic identifier and the target identifier to generate a therapeutic score value representing a link between the therapeutic identifier and the target identifier;
processing the plurality of result graphs using the therapeutic identifier, the target identifier, and the disease identifier to generate a liability score value representing a link between one or more risk tags and the therapeutic identifier;
processing the biology score value, the therapeutic score value, and the liability score value to generate combined score value representing a likelihood that a disease identified by the disease identifier is treatable with a therapeutic identified with the therapeutic identifier; and
presenting the combined score value, wherein the combined score value is displayed on a user device.

2. The method of claim 1, further comprising:
processing a plurality of files to generate the plurality of result graphs, wherein the plurality of result graphs comprises a result graph that includes a node that corresponds to text from a file describing an experiment related to one or more of the target identifier, the disease identifier, the therapeutic identifier.

3. The method of claim 1, wherein processing a plurality of result graphs to generate the biology score value comprises:
searching the plurality of result graphs for a set of target result graphs comprising the target identifier and the disease identifier; and
processing the set of target result graphs to generate the biology score value representing a likelihood that a target represented by the target identifier affects the disease represented by the disease identifier.

4. The method of claim 1, wherein processing a plurality of result graphs to generate the therapeutic score value comprises:
searching the plurality of result graphs for a set of therapeutic result graphs each comprising the therapeutic identifier and the target identifier; and
processing the set of therapeutic result graphs to generate the therapeutic score value representing a likelihood that the therapeutic represented by the therapeutic identifier affects a target represented by the target identifier.

5. The method of claim 1, wherein processing the plurality of result graphs to generate the liability score value comprises:
searching the plurality of result graphs for a set of liability result graphs linked to a set of risk tags identifying a set of risk types for a set of experiments involving the therapeutic identifier;
processing the set of liability result graphs and the set of risk tags to generate the liability score value representing a likelihood that the therapeutic represented by the therapeutic identifier is associated with an adverse event.

6. The method of claim 1, further comprising:
identifying a result graph that affects the biology score value;
processing the result graph using a language model to generate a summary of an experiment represented by the result graph; and
presenting the summary, wherein the summary is presented as a promoter summary when the result graph increases the biology score value, and wherein the summary is presented as a demoter summary when the result graph decreases the biology score value.

7. The method of claim 1, further comprising:
identifying a result graph that affects the therapeutic score value;
processing the result graph using a language model to generate a summary of an experiment represented by the result graph; and
presenting the summary, wherein the summary is presented as a promoter summary when the result graph increases the therapeutic score value, and wherein the summary is presented as a demoter summary when the result graph decreases the therapeutic score value.

8. The method of claim 1, further comprising:
identifying a result graph that affects the liability score value;
processing the result graph using a language model to generate a summary of an experiment represented by the result graph; and
presenting the summary, wherein the summary is presented as a passive summary.

9. The method of claim 1, further comprising:
processing project data and accounting data using the target identifier, the disease identifier, and the therapeutic identifier to generate a net present value for a project.

10. The method of claim 1, further comprising:
processing a plurality of combined score values, including the combined score value, to generate a timeline of combined score values; and
presenting the timeline with the combined score value.

11. A system comprising:
at least one processor;
an application executing on the at least one processor to perform:
processing a plurality of result graphs using a target identifier and a disease identifier to generate a biology score value representing a link between the target identifier and the disease identifier;
processing the plurality of result graphs using a therapeutic identifier and the target identifier to generate a therapeutic score value representing a link between the therapeutic identifier and the target identifier;
processing the plurality of result graphs using the therapeutic identifier, the target identifier, and the disease identifier to generate a liability score value representing a link between one or more risk tags and the therapeutic identifier;
processing the biology score value, the therapeutic score value, and the liability score value to generate combined score value representing a likelihood that a disease identified by the disease identifier is treatable with a therapeutic identified with the therapeutic identifier; and
presenting the combined score value, wherein the combined score value is displayed on a user device.

12. The system of claim 11, wherein the application further performs:
processing a plurality of files to generate the plurality of result graphs, wherein the plurality of result graphs comprises a result graph that includes a node that corresponds to text from a file describing an experiment related to one or more of the target identifier, the disease identifier, the therapeutic identifier.

13. The system of claim 11, wherein processing a plurality of result graphs to generate the biology score value comprises:
searching the plurality of result graphs for a set of target result graphs comprising the target identifier and the disease identifier; and
processing the set of target result graphs to generate the biology score value representing a likelihood that a target represented by the target identifier affects the disease represented by the disease identifier.

14. The system of claim 11, wherein processing a plurality of result graphs to generate the therapeutic score value comprises:
searching the plurality of result graphs for a set of therapeutic result graphs each comprising the therapeutic identifier and the target identifier; and
processing the set of therapeutic result graphs to generate the therapeutic score value representing a likelihood that the therapeutic represented by the therapeutic identifier affects a target represented by the target identifier.

15. The system of claim 11, wherein processing the plurality of result graphs to generate the liability score value comprises:
searching the plurality of result graphs for a set of liability result graphs linked to a set of risk tags identifying a set of risk types for a set of experiments involving the therapeutic identifier;
processing the set of liability result graphs and the set of risk tags to generate the liability score value representing a likelihood that the therapeutic represented by the therapeutic identifier is associated with an adverse event.

16. The system of claim 11, wherein the application further performs:
identifying a result graph that affects the biology score value;
processing the result graph using a language model to generate a summary of an experiment represented by the result graph; and
presenting the summary, wherein the summary is presented as a promoter summary when the result graph increases the biology score value, and wherein the summary is presented as a demoter summary when the result graph decreases the biology score value.

17. The system of claim 11, wherein the application further performs:
identifying a result graph that affects the therapeutic score value;
processing the result graph using a language model to generate a summary of an experiment represented by the result graph; and
presenting the summary, wherein the summary is presented as a promoter summary when the result graph increases the therapeutic score value, and wherein the summary is presented as a demoter summary when the result graph decreases the therapeutic score value.

18. The system of claim 11, wherein the application further performs:
identifying a result graph that affects the liability score value;
processing the result graph using a language model to generate a summary of an experiment represented by the result graph; and
presenting the summary, wherein the summary is presented as a passive summary.

19. The system of claim 11, wherein the application further performs:
processing project data and accounting data using the target identifier, the disease identifier, and the therapeutic identifier to generate a net present value for a project.

20. A non-transitory computer readable storage medium storing computer readable program code which, when executed by a processor, performs:
processing a plurality of result graphs using a target identifier and a disease identifier to generate a biology score value representing a link between the target identifier and the disease identifier;
processing the plurality of result graphs using a therapeutic identifier and the target identifier to generate a therapeutic score value representing a link between the therapeutic identifier and the target identifier;
processing the plurality of result graphs using the therapeutic identifier, the target identifier, and the disease identifier to generate a liability score value representing a link between one or more risk tags and the therapeutic identifier;
processing the biology score value, the therapeutic score value, and the liability score value to generate combined score value representing a likelihood that a disease identified by the disease identifier is treatable with a therapeutic identified with the therapeutic identifier; and
presenting the combined score value, wherein the combined score value is displayed on a user device.
